SEQUENCE LISTING (5 sequences)

SEQ ID NO: 1 - RNA, 4353 nt, Homo sapiens
attggaagaagaagcatagtatagaagaaaggcaaacacaacacattcaacctctgccaccatggggaactgggctgtgaatgaggggctctccatttttgtcattctggtttggctggggttgaacgtcttcctctttgtctggtattaccgggtttatgatattccacctaagttcttttacacaagaaaacttcttgggtcagcactggcactggccagggcccctgcagcctgcctgaatttcaactgcatgctgattctcttgccagtctgtcgaaatctgctgtccttcctcaggggttccagtgcgtgctgctcaacaagagttcgaagacaactggacaggaatctcacctttcataaaatggtggcatggatgattgcacttcactctgcgattcacaccattgcacatctatttaatgtggaatggtgtgtgaatgcccgagtcaataattctgatccttattcagtagcactctctgaacttggagacaggcaaaatgaaagttatctcaattttgctcgaaagagaataaagaaccctgaaggaggcctgtacctggctgtgaccctgttggcaggcatcactggagttgtcatcacgctgtgcctcatattaattatcacttcctccaccaaaaccatccggaggtcttactttgaagtcttttggtacacacatcatctctttgtgatcttcttcattggccttgccatccatggagctgaacgaattgtacgtgggcagaccgcagagagtttggctgtgcataatataacagtttgtgaacaaaaaatctcagaatggggaaaaataaaggaatgcccaatccctcagtttgctggaaaccctcctatgacttggaaatggatagtgggtcccatgtttctgtatctctgtgagaggttggtgcggttttggcgatctcaacagaaggtggtcatcaccaaggtggtcactcaccctttcaaaaccatcgagctacagatgaagaagaaggggttcaaaatggaagtgggacaatacatttttgtcaagtgcccaaaggtgtccaagctggagtggcacccttttacactgacatccgcccctgaggaagacttctttagtatccatatccgcatcgttggggactggacagaggggctgttcaatgcttgtggctgtgataagcaggagtttcaagatgcgtggaaactacctaagatagcggttgatgggccctttggcactgccagtgaagatgtgttcagctatgaggtggtgatgttagtgggagcagggattggggtcacacccttcgcatccattctcaagtcagtctggtacaaatattgcaataacgccaccaatctgaagctcaaaaagatctacttctactggctgtgccgggacacacatgcctttgagtggtttgcagatctgctgcaactgctggagagccagatgcaggaaaggaacaatgccggcttcctcagctacaacatctacctcactggctgggatgagtctcaggccaatcactttgctgtgcaccatgatgaggagaaagatgtgatcacaggcctgaaacaaaagactttgtatggacggcccaactgggataatgaattcaagacaattgcaagtcaacaccctaataccagaataggagttttcctctgtggacctgaagccttggctgaaaccctgagtaaacaaagcatctccaactctgagtctggccctcggggagtgcatttcattttcaacaaggaaaacttctaacttgtctcttccatgaggaaataaatgtgggttgtgctgccaaatgctcaaataatgctaattgataatataaataccccctgcttaaaaatggacaaaaagaaactataatgtaatggttttcccttaaaggaatgtcaaagattgtttgatagtgataagttacatttatgtggagctctatggttttgagagcacttttacaaacattatttcatttttttcctctcagtaatgtcagtggaagttagggaaaagattcttggactcaattttagaatcaaaagggaaaggatcaaaaggttcagtaacttccctaagattatgaaactgtgaccagatctagcccatcttactccaggtttgatactctttccacaatactgagctgcctcagaatcctcaaaatcagtttttatattccccaaaagaagaaggaaaccaaggagtagctatatatttctactttgtgtcatttttgccatcattattatcatactgaaggaaattttccagatcattaggacataatacatgttgagagtgtctcaacacttattagtgacagtattgacatctgagcatactccagtttactaatacagcagggtaactgggccagatgttctttctacagaagaatattggattgattggagttaatgtaatactcatcatttaccactgtgcttggcagagagcggatactcaagtaagttttgttaaatgaatgaatgaatttagaaccacacaatgccaagatagaattaatttaaagccttaaacaaaatttatctaaagaaataacttctattactgtcatagaccaaaggaatctgattctccctagggtcaagaacaggctaaggatactaaccaataggattgcctgaagggttctgcacattcttatttgaagcatgaaaaaagagggttggaggtggagaattaacctcctgccatgactctggctcatctagtcctgctccttgtgctataaaataaatgcagactaatttcctgcccaaagtggtcttctccagctagcccttatgaatattgaacttaggaattgtgacaaatatgtatctgatatggtcatttgttttaaataacacccaccccttattttccgtaaatacacacacaaaatggatcgcatctgtgtgactaatggtttatttgtattatatcatcatcatcatcctaaaattaacaacccagaaacaaaaatctctatacagagatcaaattcacactcaatagtatgttctgaatatatgttcaagagagagtctctaaatcactgttagtgtggccaagagcagggttttctttttgttcttagaactgctcccatttctgggaactaaaaccagttttatttgccccaccccttggagccacaaatgtttagaactcttcaacttcggtaatgaggaagaaggagaaagagctgggggaagggcagaagactggtttaggaggaaaaggaaataaggagaaaagagaatgggagagtgagagaaaataaaaaaggcaaaagggagagagaggggaagggggtctcatattggtcattccctgccccagatttcttaaagtttgatatgtatagaatataattgaaggaggtatacacatattgatgttgttttgattatctatggtattgaatcttttaaaatctggtcacaaattttgatgctgagggggattattcaagggactaggatgaactaaataagaactcagttgttctttgtcatactactattcctttcgtctcccagaatcctcagggcactgagggtaggtctgacaaataaggcctgctgtgcgaatatagcctttctgaaatgtaccaggatggtttctgcttagagacacttaggtccagcctgttcacactgcacctcaggtatcaattcatctattcaacagatatttattgtgttattactatgagtcaggctctgtttattgtttcaattctttacaccaaagtatgaactggagagggtacctcagttataaggagtctgagaatattggccctttctaacctatgtgcataattaaaaccagcttcatttgttgctccgagagtgtttctccaaggttttctatcttcaaaaccaactaagttatgaaagtagagagatctgccctgtgttatccagttatgagataaaaaatgaatataagagtgcttgtcattataaaagtttccttttttattctctcaagccaccagctgccagccaccagcagccagctgccagcctagctttttttttttttttttttttttagcacttagtatttagcatttattaacaggtactctaagaatgatgaagcattgtttttaatcttaagactatgaaggtttttcttagttcttctgcttttgcaattgtgtttgtgaaatttgaatacttgcaggctttgtatgtgaataattctagcgggggacctgggagataattcctacggggaattcttaaaactgtgctcaactattaaaatgaatgagctttcaaaaaaaaaaaaaaaaaaaaaaaaaaaaaaaaaaa

SEQ ID NO: 2 - DNA, 25 bp, Homo sapiens
agaagtaagtggaagggcccagaag

SEQ ID NO: 3 - DNA, 25 bp, Homo sapiens
agggaaactgggagaggagaaatat

SEQ ID NO: 4 - DNA, 25 bp, Homo sapiens
tcagcgcaggggcgcccggttcttt

SEQ ID NO: 5 - DNA, 25 bp, Homo sapiens
atcgacaagaccggcttccatccga

(19) United States
(12) Patent Application Publication
(10) Pub. No.: US 2020/0399642 A1
(43) Pub. Date: Dec. 24, 2020

(54) METHODS AND COMPOSITIONS FOR TREATING AND PREVENTING METASTATIC TUMORS

(71) Applicant: IMMUNE PHARMACEUTICALS, INC., ENGLEWOOD CLIFFS, NJ (US)

(72) Inventors: Anna Martner, Gothenburg (SE); Kristoffer Hellstrand, Gothenburg (SE)

(21) Appl. No.: 16/773,094

(22) Filed: Jan. 27, 2020

Related U.S. Application Data
(63) Continuation of application No. PCT/US2018/043693, filed on Jul. 25, 2018.
(60) Provisional application No. 62/537,895, filed on Jul. 27, 2017.

(51) Int. Cl. C12N 15/113; A61P 35/04; A61K 31/417; C12N 15/11; A61K 38/20; A61K 38/21; A61K 31/7088

(52) U.S. Cl. CPC C12N 15/1137; A61P 35/04; A61K 31/417; C12N 15/11; C12N 2320/31; A61K 38/217; A61K 31/7088; C12N 2310/11; A61K 38/2086

(57) ABSTRACT

Some embodiments of the methods and compositions provided herein relate to the treatment and amelioration of metastatic tumors and to the prevention of distant metastasis. In some embodiments, a metastatic tumor, such as a melanoma, can be treated by reducing the activity of NOX2 in a cell of a subject. In some embodiments, the activity of NOX2 can be reduced by administering a NOX2 inhibitor, such as histamine dihydrochloride (HDC).

### CROSS-REFERENCE TO RELATED APPLICATIONS

This application claims priority to U.S. Prov. App. No. 62/537,895 filed Jul. 27, 2017 entitled “NOX2 IN REGULATION OF MELANOMA METASTASIS”, the content of which is incorporated herein by reference in its entirety.

### REFERENCE TO SEQUENCE LISTING

The present application is being filed along with a Sequence Listing in electronic format. The Sequence Listing is provided as a file entitled IMMUN233WOSEQLIST, created Jul. 18, 2018, which is approximately 6 Kb in size. The information in the electronic format of the Sequence Listing is incorporated herein by reference in its entirety.

### FIELD

Some embodiments of the methods and compositions provided herein relate to the prevention, treatment and amelioration of metastatic tumors. In some embodiments, a metastatic tumor, such as a melanoma, can be prevented or treated by reducing the activity of NOX2 in a cell of a subject. In some embodiments, the activity of NOX2 can be reduced by administering a NOX2 inhibitor, such as histamine dihydrochloride (HDC).

### BACKGROUND

Metastatic cancer is the spread of a cancer from one organ to another organ or another site in a subject. Metastasis is a complex series of biological steps in which cancerous cells migrate from an original site to another site in a subject. Once a cancer has metastasized, the treatment of metastatic cancer relies on the same traditional techniques to treat primary cancer, such as radiosurgery, chemotherapy, radiation therapy and surgery, immunotherapy, or a combination of these interventions. In many cases, currently available therapies are not able to cure the metastatic cancer, although metastatic testicular cancer and thyroid cancer are notable exceptions. In addition, few current therapies are available to prevent the metastatic spread of cancer cells.

Metastatic cancer is also of particular concern as the incidence of some cancers, such as melanoma and breast cancer, remains high in younger people resulting in a profound effect on the number of productive years lost due to the illness. Melanoma, for example, is a cancer that has a very high incidence and mortality rate. In some countries, melanoma is the most common type of cancer in young adults. The mean age at diagnosis of melanoma is around 50 years, which is 10-15 years earlier than the commoner diagnoses of prostate, bowel, and lung cancer. Therefore, melanoma is second only to breast cancer in the number of productive years lost. A primary melanoma is typically removed surgically, but several patients will develop nodal or distant metastasis despite the removal of the primary tumor. The 15-year survival rates for localized melanoma exceed 50%, but fall to 30% when there is nodal involvement. Melanoma with distant metastasis is associated with poor survival. The past decade has seen the development of immunotherapy that results in durable regression of metastatic tumors in a minority of patients with melanoma or other forms of cancer. However, there remains a high need for additional treatments that ameliorate melanoma metastases and for treatments that prevent metastasis of the primary cancer.

Single-agent dacarbazine chemotherapy, with modest response rates of 15-20%, has, until recently, been the standard of care for treatment of metastatic melanoma as no combination chemotherapy has previously been demonstrated to have an improved overall survival in phase III randomized controlled trials. An additional complication with respect to the treatment of both primary and metastatic cancers is that treatment regimens involving standard chemotherapeutic agents are known to have variable and unpredictable effects, including efficacy and the extent of undesired side effects. Therefore, there is a need to provide improved methods and compositions for the prevention, treatment and amelioration of metastatic cancers, such as metastatic melanoma.

### SUMMARY

Some embodiments of the methods and compositions provided herein include a method of preventing metastasis of a primary tumor in a subject, or treating or ameliorating a metastatic tumor in a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject.

In some embodiments, reducing the activity of NOX2 comprises administering an effective amount of a NOX2 inhibitor to the subject. In some embodiments, the NOX2 inhibitor is selected from the group consisting of histamine dihydrochloride (HDC), histamine, N-methyl-histamine, 4-methyl-histamine, histamine phosphate, histamine diphosphate, GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, NSC23766, CAS 1177865-17-6, and CAS 1090893-12-1, and shionogi. In some embodiments, the NOX2 inhibitor is HDC.

In some embodiments, reducing the expression level of a nucleic acid encoding NOX2 or the expression level of NOX2 protein in a cell comprises contacting the cell with an isolated nucleic acid selected from the group consisting of a guide RNA (gRNA), a small hairpin RNA (shRNA), a small interfering RNA (siRNA), a micro RNA (miRNA), an antisense polynucleotide, and a ribozyme. In some embodiments, the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof. In some embodiments, the isolated nucleic acid comprises a gRNA comprising a sequence complementary to the sequence of a target gene selected from the group consisting of NOX2, CYBA, NCF1, NCF2, NCF4, RAC1, and RAC2. In some embodiments, the target gene is NOX2.

Some embodiments also include administering an additional therapeutic agent in combination with the NOX2 inhibitor or the isolated nucleic acid. In some embodiments, the additional therapeutic agent is a NK cell activating agent. In some embodiments, the NK cell activating agent is selected from the group consisting of IL-15, IFN-γ, IL-12, IL-18, IL-2, and CCL5. In some embodiments, the additional therapeutic agent is IL-15. In some embodiments, the additional therapeutic agent is IFN-γ. In some embodiments, the additional therapeutic agent and the NOX2 inhibitor or the isolated nucleic acid are administered sequentially. In some embodiments, the additional therapeutic agent and the NOX2 inhibitor or the isolated nucleic acid are administered concurrently.

In some embodiments, the primary or metastatic tumor is selected from the group consisting of a melanoma, a bladder cancer, a breast cancer, a pancreatic cancer, a colorectal cancer, a renal cancer, a prostate cancer, a stomach cancer, a thyroid cancer, a uterine cancer, and an ovarian cancer. In some embodiments, the metastatic tumor comprises a melanoma. In some embodiments, the melanoma is selected from the group consisting of lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, and vaginal melanoma. In some embodiments, the primary or metastatic tumor is located at a site selected from the group consisting of lung, liver, brain, peritoneum, adrenal gland, skin, muscle, vagina, and bone. In some embodiments, the metastatic tumor is located in a lung.

In some embodiments, the cell is a hematopoietic cell. In some embodiments, the cell is a myeloid cell.

Some embodiments also include identifying the metastatic tumor in the subject.

In some embodiments, the subject is mammalian. In some embodiments, the subject is human.

Some embodiments of the methods and compositions provided herein include a method of increasing the level of natural killer (NK) cells in a metastatic tumor of a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject, wherein the level of NK cells in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

Some embodiments of the methods and compositions provided herein include a method of decreasing the level of reactive oxygen species (ROS) in a metastatic tumor of a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject, wherein the level of ROS in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

In some embodiments, reducing the activity of NOX2 comprises administering an effective amount of a NOX2 inhibitor to the subject. In some embodiments, the NOX2 inhibitor is selected from the group consisting of histamine dihydrochloride (HDC), histamine, N-methyl-histamine, 4-methyl-histamine, histamine phosphate, histamine diphosphate, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, NSC23766, CAS 1177865-17-6, and CAS 1090893-12-1, and shionogi. In some embodiments, the NOX2 inhibitor is HDC.

In some embodiments, reducing the expression level of a nucleic acid encoding NOX2 or the expression level of NOX2 protein in a cell comprises contacting the cell with an isolated nucleic acid selected from the group consisting of a guide RNA (gRNA), a small hairpin RNA (shRNA), a small interfering RNA (siRNA), a micro RNA (miRNA), an antisense polynucleotide, and a ribozyme. In some embodiments, the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof. In some embodiments, the isolated nucleic acid comprises a gRNA comprising a sequence complementary to the sequence of a target gene selected from the group consisting of NOX2, CYBA, NCF1, NCF2, NCF4, RAC1, and RAC2. In some embodiments, the target gene is NOX2.

Some embodiments also include administering an additional therapeutic agent in combination with the NOX2 inhibitor or the isolated nucleic acid. In some embodiments, the additional therapeutic agent is a NK cell activating agent. In some embodiments, the NK cell activating agent is selected from the group consisting of IL-15, IFN-γ, IL-12, IL-18, IL-2, and CCL5. In some embodiments, the additional therapeutic agent is IL-15. In some embodiments, the additional therapeutic agent is IFN-γ. In some embodiments, the additional therapeutic agent and the NOX2 inhibitor or the isolated nucleic acid are administered sequentially. In some embodiments, the additional therapeutic agent and the NOX2 inhibitor or the isolated nucleic acid are administered concurrently.

In some embodiments, the primary or metastatic tumor is selected from the group consisting of a melanoma, a bladder cancer, a breast cancer, a pancreatic cancer, a colorectal cancer, a renal cancer, a prostate cancer, a stomach cancer, a thyroid cancer, a uterine cancer, and an ovarian cancer. In some embodiments, the metastatic tumor comprises a melanoma. In some embodiments, the melanoma is selected from the group consisting of lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, and vaginal melanoma. In some embodiments, the primary or metastatic tumor is located at a site selected from the group consisting of lung, liver, brain, peritoneum, adrenal gland, skin, muscle, vagina, and bone. In some embodiments, the metastatic tumor is located in a lung,

In some embodiments, the cell is a hematopoietic cell. In some embodiments, the cell is a myeloid cell.

Some embodiments also include identifying the metastatic tumor in the subject.

In some embodiments, the subject is mammalian. In some embodiments, the subject is human

Some embodiments of the methods and compositions provided herein include a use of a nicotinamide adenine dinucleotide phosphate oxidase 2 (NOX2) inhibitor or an isolated nucleic acid to prevent metastasis of a primary tumor, or treat or ameliorate a metastatic tumor in a subject, wherein the isolated nucleic acid reduces the expression level of a nucleic acid encoding NOX2 or the expression level of NOX2 protein in a cell of the subject.

Some embodiments of the methods and compositions provided herein include use of a nicotinamide adenine dinucleotide phosphate oxidase 2 (NOX2) inhibitor or an isolated nucleic acid to increase the level of natural killer (NK) cells in a metastatic tumor of a subject, wherein the isolated nucleic acid reduces the expression level of a nucleic acid encoding NOX2 or the expression level of NOX2 protein in a cell of the subject. In some embodiments, the level of NK cells in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

Some embodiments of the methods and compositions provided herein include use of a nicotinamide adenine dinucleotide phosphate oxidase 2 (NOX2) inhibitor or an isolated nucleic acid to decrease the level of reactive oxygen species (ROS) in a metastatic tumor of a subject, wherein the isolated nucleic acid reduces the expression level of a nucleic acid encoding NOX2 or the expression level of NOX2 protein in a cell of the subject. In some embodiments, the level of ROS in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

In some embodiments, the NOX2 inhibitor is selected from the group consisting of histamine dihydrochloride (HDC), histamine, N-methyl-histamine, 4-methyl-histamine, histamine phosphate, histamine diphosphate, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, NSC23766, CAS 1177865-17-6, and CAS 1090893-12-1, and shionogi. In some embodiments, the NOX2 inhibitor is HDC.

In some embodiments, the isolated nucleic acid is selected from the group consisting of a guide RNA (gRNA), a small hairpin RNA (shRNA), a small interfering RNA (siRNA), a micro RNA (miRNA), an antisense polynucleotide, and a ribozyme. In some embodiments, the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof. In some embodiments, the isolated nucleic acid comprises a gRNA comprising a sequence complementary to the sequence of a target gene selected from the group consisting of NOX2, CYBA, NCF1, NCF2, NCF4, RAC1, and RAC2. In some embodiments, the target gene is NOX2.

In some embodiments, the use is in combination with an additional therapeutic agent. In some embodiments, the additional therapeutic agent is a NK cell activating agent. In some embodiments, the NK cell activating agent is selected from the group consisting of IL-15, IFN-γ, IL-12, IL-18, IL-2, and CCL5. In some embodiments, the additional therapeutic agent is IL-15. In some embodiments, the additional therapeutic agent is IFN-γ.

In some embodiments, the primary or metastatic tumor is selected from the group consisting of a melanoma, a bladder cancer, a breast cancer, a pancreatic cancer, a colorectal cancer, a renal cancer, a prostate cancer, a stomach cancer, a thyroid cancer, a uterine cancer, and an ovarian cancer. In some embodiments, the metastatic tumor comprises a melanoma. In some embodiments, the melanoma is selected from the group consisting of lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, and vaginal melanoma. In some embodiments, the primary or metastatic tumor is located at a site selected from the group consisting of lung, liver, brain, peritoneum, adrenal gland, skin, muscle, vagina, and bone. In some embodiments, the metastatic tumor is located in a lung.

In some embodiments, the cell is a hematopoietic cell. In some embodiments, the cell is a myeloid cell.

In some embodiments, the subject is mammalian. In some embodiments, the subject is human

### BRIEF DESCRIPTION OF THE DRAWINGS

FIGS. 1A-1C depict the impact of genetic and pharmacologic inhibition of NOX2 on B16 melanoma metastasis. FIG. 1A depicts an experimental design. FIG. 1B is a graph depicting the number of metastatic foci formed in lungs of wild-type (WT) and Nox2-KO (Nox2^{-/-}) mice. Medians and quartiles are indicated by boxes. Error bars show the min to max values (n=6 for each group; t test; two independent experiments). FIG. 1C is a graph depicting the number of metastatic foci in lungs of WT or Nox2-KO mice after systemic treatment with HDC and/or IL15. FIG. 1D is a graph depicting the results from lung metastasis formation by the B16F1 melanoma cell line following similar treatment. The results shown in FIGS. 1C-D were evaluated by repeated measures for analysis of variance (ANOVA). P>0.05; *, P≤0.05; **, P≤0.01; ***, P≤0.001.

FIGS. 2A-2G depict the effect of HDC on the ROS production in mouse lungs after melanoma cell inoculation. FIG. 2A is a graph depicting the extracellular ROS production of PMA-stimulated WT Gr1⁺ (solid line), WT Gr1⁻ (dashed line), or Nox2^{-/-} Gr1⁺ mouse lung cells (line following zero x-axis) in a representative experiment out of three performed. FIG. 2B is a graph depicting the ROS production of WT lung cells triggered by 10⁻⁷ M WKYMVm, following inhibition by HDC at indicated final concentrations. The mean ROS production ±SEM is displayed. The degree of inhibition exerted by HDC was analyzed by t-test (n=3). FIG. 2C depicts an experimental design for assessment of the dynamics of ROS-producing myeloid cells in lungs after B16 cell inoculation. FIG. 2D is a representative dot plot of CD11b⁺Gr1⁺ cells out of live CD45⁺ lung cells before (0 hour) or 0.5 hours after tumor cell inoculation. FIG. 2E is a graph depicting the fraction of CD11b⁺Gr1⁺ cells out of live CD45⁺ cells in lungs at indicated time points after tumor cell injection, with or without pretreatment of mice with PBS (control) or HDC 24 hours before tumor cell injection, as determined in single-cell lung suspensions (n=18 in each group; four independent experiments). FIG. 2F is a graph depicting the ROS formation (area under curve, AUC) ex vivo in response to PMA stimulation of single lung cell suspensions from mice pretreated with HDC or PBS on the day before tumor cell inoculation. ROS production was determined at 30 minutes and 24 hours after inoculation of 100,000 B16 cells (n=5-10, t test; three independent experiments). FIG. 2G is a graph depicting the reduced B16F10 metastasis formation and lack of effects of systemic treatment with HDC on metastasis formation in animals depleted of Gr1+ cells prior to melanoma cell inoculation (n=4-5 for each group, one way ANOVA). Nonsignificant values: n.s; P>0.05; *, P≤0.05; **, P≤0.01; ***, P≤0.001.

FIGS. 3A-3C depict that the antimetastatic effects of HDC rely on natural killer (NK) cells and NK cell-derived IFNγ. FIG. 3A is a graph depicting the effects of systemic treatment with HDC on B16F10 metastasis formation in WT and Nox2^{-/-} animals depleted of NK cells (n=7 for untreated WT mice with and without NK cells (two independent experiments); n=3 for HDC-treated WT mice with and without NK cells; n=4 for each group of Nox2^{-/-} mice, one way ANOVA). FIG. 3B is a graph depicting the effects of systemic treatment with HDC on NK-cell numbers in lungs and spleens of WT and Nox-KO (Nox2^{-/-}) mice at 3 weeks after tumor cell inoculation. The percentage of NK cells out of live CD45⁺ cells was determined by flow cytometry (WT mice n=9-11; Nox2^{-/-} mice n=9-13, t test; three independent experiments). FIG. 3C is a graph depicting IFNγ levels produced in lung cells from HDC-treated control WT mice and NK-cell depleted mice (▴, NK-dep). Mice received HDC (▪) or PBS (●, control) 24 hours before i.v. inoculation of B16 cells. Lungs were recovered 30 minutes after tumor cell inoculation and were then incubated in vitro with B16 melanoma cells at indicated effector to target cell ratios, after which IFNγ levels in the cultures were determined (n=11 for the control group, n=6 for the other groups, two-way ANOVA; two independent experiments). Nonsignificant values: n.s.; P>0.05; *, P≤0.05; **, P≤0.01; ***, P≤0.001.

FIGS. 4A-4C depict impact of IFNγ in B16 melanoma metastasis. FIG. 4A is a graph of box plots of B16F10 metastasis at 3 weeks after i.v. inoculation of 50,000, 100,000, or 150,000 B16 melanoma cells into WT and Ifng^{-/-} mice (n=6 for each group, t test; two independent experiments). FIG. 4B is a graph in which the left portion depict a lack of efficacy of systemic treatment with HDC on metastasis formation at 3 weeks after inoculation of B16 melanoma cells into Ifng^{-/-} mice (n=19-21, t test; four independent experiments); and in which the right portion depicts effects of systemic treatment with HDC on metastasis formation (% of control) in Ifng^{-/-} mice that received the adoptive transfer of purified NK cells from WT mice (n=9 for each group, t test) or purified NK cells from Ifng^{-/-} mice (n=6, t test; two independent experiments). FIG. 4C is a photograph of an autoradiograph depicting the presence of WT Ifng in peripheral blood collected from six representative Ifng^{-/-} mice who had received adoptive transfer of WT NK cells (lanes 1-3) or Ifng^{-/-} NK cells (lanes 4-6) 2 days earlier. PCR was performed for WT Ifng and the disrupted IFNγ gene of Ifng^{-/-} mice. Nonsignificant values: n.s.; P>0.05; *, P≤0.05; **, P≤0.01; ***, P≤0.001.

### DETAILED DESCRIPTION

Some embodiments of the methods and compositions provided herein relate to the prevention metastatic tumors, and treatment and amelioration of metastatic tumors. In some embodiments, a metastatic tumor, such as a melanoma, can be prevented or treated by reducing the activity of NOX2 in a cell of a subject. In some embodiments, the activity of NOX2 can be reduced by administering a NOX2 inhibitor, such as histamine dihydrochloride (HDC). In some embodiments, the activity of NOX2 can be reduced by administering an isolated nucleic which reduces the expression level of a nucleic acid encoding NOX2 or the expression level of NOX2 protein in a cell of the subject.

The NADPH oxidase 2 of myeloid cells, NOX2, generates reactive oxygen species (ROS) to eliminate pathogens and malignant cells. NOX2-derived ROS have also been proposed to dampen functions of natural killer (NK) cells and other antineoplastic lymphocytes in the microenvironment of established tumors. The NOX2 protein is encoded by the CYBB gene and forms a holoenzyme that can include other proteins such as cytochrome b alpha encoded by a CYBA gene, and can include regulatory subunits p67phox, p47phox, p40phox, Rac1, and Rac2. The mechanisms by which NOX2 and ROS influence the process of distant metastasis are not well understood. Embodiments described herein include the use of genetically NOX2-deficient mice and pharmacologic inhibition of NOX2 to elucidate the role of NOX2 for the hematogenous metastasis of melanoma cells. After intravenous inoculation of B16F1 or B16F10 cells, lung metastasis formation was reduced in B6.129S6-Cybb^{tm1DinK} (Nox2-KO) versus Nox2-sufficient wild-type (WT) mice. Systemic treatment with the NOX2-inhibitor, HDC, reduced melanoma metastasis and enhanced the infiltration of IFNγ-producing NK cells into lungs of WT but not of Nox2-KO mice. IFNγ-deficient B6.129S7-Ifng^{tm1Ts}/J mice were prone to develop melanoma metastases and did not respond to in vivo treatment with HDC. NOX2-derived ROS may facilitate metastasis of melanoma cells by downmodulating NK-cell function. Thus, inhibition of NOX2 may restore IFNγ-dependent, NK cell-mediated clearance of melanoma cells (Aydin, E. et al., (2017) “Role of NOX2-Derived Reactive Oxygen Species in NK Cell-Mediated Control of Murine Melanoma Metastasis”, Cancer Immunol Res 5 (9) 804-811, which is incorporated by reference in its entirety).

Reactive oxygen species (ROS) are short-lived compounds that arise from electron transfer across biological membranes where the electron acceptor is molecular oxygen and the initial product is superoxide anion (O₂⁻). ROS refer to oxygen radicals such as O₂⁻ and the hydroxyl radical (OH.) along with nonradicals, including hydrogen peroxide, that share the oxidizing capacity of oxygen radicals and may be converted into radicals. ROS are generated as by-products of mitochondrial ATP generation in the electron transport chain but are also produced in a regulated fashion by the NADPH oxidases (NOX) and the dual oxidases (DUOX). This family of transmembrane proteins comprises NOX 1-5 and DUOX 1-2, whose only known function is to produce ROS.

The NOX proteins are structurally similar and utilize a similar principal mechanism of ROS generation but vary in cellular and subcellular distribution. NOX2 is expressed almost exclusively in cells of the myeloid lineage such as monocyte/macrophages and neutrophilic granulocytes. These cells utilize NOX2-derived ROS to eliminate intra- and extracellular microorganisms. NOX2 has also been linked to immunosuppression in cancer: when released from myeloid cells into the extracellular space, ROS generated by NOX2 may trigger dysfunction and apoptosis of adjacent antineoplastic lymphocytes, including NK cells. The strategy to target ROS formation by myeloid cells has been proposed to improve the efficiency of cancer immunotherapy.

The role of ROS and NOX2 for the growth and metastatic spread of cancer cells is, however, complex and controversial. Thus, although the genetic disruption of Nox2 reduces the subcutaneous growth of murine melanoma and lung carcinoma, it does not affect sarcoma growth or prostate cancer growth in mice. Also, the in vivo administration of scavengers of ROS such as N-acetyl-cysteine reduces the tumorigenicity of murine melanoma cells but enhances lymph node metastasis in other melanoma models, accelerates tumor progression in mouse models of B-RAF- and K-RAS-induced lung cancer and accelerates the metastasis of xenografted human melanoma cells in immunodeficient mice.

The detailed mechanisms of relevance to the discrepant impact of ROS for the growth and spread of cancer cells remain to be elucidated. Further understanding of the role of ROS for cancer progression requires experimental models that address a distinct phase of tumor progression, define the source of ROS, and take mechanisms of immuno-surveillance into account. Some embodiments described herein include determining the impact of genetic and pharmacologic inhibition of NOX2 in a murine NK cell-dependent model of melanoma metastasis.

### Methods of Treatment

Some embodiments of the methods and compositions provided herein include preventing, treating or ameliorating a subject having a disorder, such as preventing metastasis of a primary tumor, and treating or ameliorating a metastatic tumor. As used herein, “subject” can include a human or a non-human mammal, e.g., a dog, a cat, a mouse, a rat, a cow, a sheep, a pig, a goat, a non-human primate or a bird, e.g., a chicken, as well as any other vertebrate or invertebrate. As used herein, “treat,” “treatment,” or “treating,” can include administering a pharmaceutical composition to a subject for therapeutic purposes, and can include reducing the symptoms or consequences of a disorder, such as preventing the occurrence of metastases from a primary tumor, reducing the number of tumor cells of a metastatic tumor or inhibiting the growth of tumor cells of a metastatic tumor; and can include curing a disorder, such as eliminating the symptoms of a disorder, such as the elimination of tumor cells of a metastatic tumor in a subject. As used herein, “ameliorate”, or “ameliorating” can include a therapeutic effect which relieves, to some extent, one or more of the symptoms of a disorder. As used herein, “prevent,” “preventing” and “prevention” can include inhibiting the occurrence of a disorder, such as inhibiting the metastasis of a primary tumor, and can include preventing a primary an action that occurs before a subject begins to suffer from the regrowth of the cancer and/or which inhibits or reduces the severity of the cancer. As used herein, an “effective amount” can include an amount, such as a dose, of a therapeutic compound sufficient to treat a disorder. As used herein, reducing the activity of NOX2 can include reducing the activity of NADPH oxidase 2, and/or reducing the activity of a NADPH oxidase holoenzyme which includes the NOX2 protein.

Some embodiments include reducing the activity of NOX2 by contacting a cell with a NOX2 inhibitor. In some embodiments, the cell is a hematopoietic cell. Hematopoietic cells include myeloid cells and lymphoid cells. In some embodiments, the cell is a myeloid cell. Examples of myeloid cells include monocytes, macrophages, neutrophils, basophils, eosinophils, erythrocytes, and megakaryocytes to platelets. In some embodiments, the cell is a CD11b+ myeloid cell. In some embodiments, the cell is a lymphoid cell. Examples of lymphoid cells include T cells, B cells, and NK cells.

In some embodiments, an effective amount of a NOX2 inhibitor can be administered to a subject in need thereof. Examples of NOX2 inhibitors include histamine dihydrochloride (HDC) (CEPLENE), GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, and shionogi. Altenhofer, S. et al., “Evolution of NADPH Oxidase Inhibitors: Selectivity and Mechanisms for Target Engagement”, Antioxid Redox Signal. 2015 23: 406-427; Hirano, K. et al., “Discovery of GSK2795039, a Novel Small Molecule NADPH Oxidase 2 Inhibitor”, Antioxid Redox Signal. 2015 23: 358-374, which are each incorporated by reference in its entirety. More examples of NOX2 inhibitors include histamine, N-methyl-histamine, 4-methyl-histamine, histamine phosphate, and histamine diphosphate. In some embodiments, the NOX2 inhibitor is HDC.

In some embodiments, a NOX2 inhibitor can include RAC1 inhibitors and RAC2 inhibitor, such as NSC23766, CAS 1177865-17-6, and CAS 1090893-12-1. RAC1 and RAC2 can each be associated with NOX2 holoenzyme, and inhibition of RAC1 or RAC 2 can inhibit NOX2. See e.g., Veluthakal R., et al., (2016) “NSC23766, a Known Inhibitor of Tiam1-Rac1 Signaling Module, Prevents the Onset of Type 1 Diabetes in the NOD Mouse Model” Cell Physiol Biochem 39:760-767; and Cifuentes-Pagano, E., et al., (2014) “The Quest for Selective Nox Inhibitors and Therapeutics: Challenges, Triumphs and Pitfalls” Antioxid Redox Signal. 20: 2741-2754, which are each incorporated by reference in its entirety. More examples of RAC1 inhibitors are disclosed in Arnst, J. L. et al., (2017) “Discovery and characterization of small molecule Rac1 inhibitors”, Oncotarget. 8: 34586-34600.

### Reducing Expression Levels of NOX2

Some embodiments of the methods and compositions provided herein include reducing the activity of NOX2 in a cell by reducing the expression level of a nucleic acid encoding NOX2, or the expression level of a NOX2 protein in the cell. In some embodiments, the cell is a hematopoietic cell. In some embodiments, the cell is a myeloid cell. In some embodiments, the cell is a lymphoid cell. Some embodiments include reducing the expression level of a nucleic acid encoding NOX2, or the expression level of a NOX2 protein in a cell using either RNA interference, RNA antisense technologies or a CRISPR based system, such as a CRISPR/Cas9 system.

Some embodiments include reducing the expression level of a nucleic acid encoding NOX2, or the expression level of a NOX2 protein in a cell using a CRISPR based system, such as a CRISPR/Cas9 system. In some embodiments, a CRISPR (clustered regularly interspaced short palindromic repeats) system can be used to modify a cell to reduce the expression level of a nucleic acid encoding NOX2, or the expression level of a NOX2 protein in the cell. For example, a cell can be modified such that a target gene, such as NOX2 gene, can be functionally knocked-out. In some embodiments, a cell can be obtained from a subject. In some embodiments, the cell can be modified by a CRISPR system ex vivo. In some embodiments, the modified cell can be delivered to a subject. Examples of CRISPR systems useful with the methods and compositions provided herein are disclosed in U.S. Pat. App. Pub. 20180201951, U.S. Pat. App. Pub. 20180177893, and U.S. Pat. App. Pub. 20180105834 which are each incorporated by reference in its entirety.

A CRISPR system includes a microbial nuclease system involved in defense against invading phages and plasmids that provides a form of acquired immunity. CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a memory of past exposures. Cas9 forms a complex with the 3′ end of the sgRNA, and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Type II systems have differing PAM requirements. The Streptococcus pyogenes CRISPR system may have the PAM sequence for this Cas9 (SpCas9) as 5′-NRG-3′, where R is either A or G, and characterized the specificity of this system in human cells. A unique capability of the CRISPR/Cas9 system is the straightforward ability to simultaneously target multiple distinct genomic loci by co-expressing a single Cas9 protein with two or more sgRNAs. For example, the S. pyogenes Type II system naturally prefers to use an “NGG” sequence, where “N” can be any nucleotide, but also accepts other PAM sequences, such as “NAG” in engineered systems (Hsu et al., Nature Biotechnology (2013) doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods (2013) doi:10.1038/nmeth.2681).

An engineered form of the Type II effector system of Streptococcus pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in genome editing. The CRISPR/Cas9-based engineered systems may be designed to target any gene, such as a gene encoding NOX2. The CRISPR/Cas9-based systems may include a Cas9 protein or Cas9 fusion protein and at least one gRNA. The Cas9 fusion protein may, for example, include a domain that has a different activity that what is endogenous to Cas9, such as a transactivation domain.

The CRISPR/Cas9-based system may include a Cas9 protein or a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. The Cas9 protein may be mutated so that the nuclease activity is inactivated. An inactivated Cas9 protein from Streptococcus pyogenes (iCas9, also referred to as “dCas9”) with no endonuclease activity has been recently targeted to genes in bacteria, yeast, and human cells by gRNAs to silence gene expression through steric hindrance. As used herein, “iCas9” and “dCas9” can include a Cas9 protein that has the amino acid substitutions D10A and H840A and has its nuclease activity inactivated.

The CRISPR/Cas9-based system may include a fusion protein. The fusion protein may comprise two heterologous polypeptide domains, wherein the first polypeptide domain comprises a Cas protein and the second polypeptide domain has nuclease activity that is different from the nuclease activity of the Cas9 protein. The fusion protein may include a Cas9 protein or a mutated Cas9 protein, as described above, fused to a second polypeptide domain that has nuclease activity. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases are deoxyribonuclease and ribonuclease.

In some embodiments, a gRNA provides the targeting of the CRISPR/Cas9-based system. The gRNA is a fusion of two noncoding RNAs: a crRNA and a tracrRNA. The gRNA may target any desired DNA sequence, such as a DNA sequence encoding a NOX2 protein, by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid. The “target region”, “target sequence” or “protospacer” as used interchangeably herein refers to the region of the target gene to which the CRISPR/Cas9-based system targets. The CRISPR/Cas9-based system may include at least one gRNA, wherein the gRNAs target different DNA sequences. The target DNA sequences may be overlapping. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide.

The gRNA may target any nucleic acid sequence such as an endogenous gene, such as a NOX2 gene. The CRISPR/Cas9-based system may use gRNA of varying sequences and lengths. The gRNA may comprise a complementary polynucleotide sequence of the target DNA sequence followed by a PAM sequence. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. The PAM sequence may be “NGG”, where “N” can be any nucleotide. The gRNA may target at least one of the promoter region, the enhancer region or the transcribed region of the target gene.

In some embodiments, a target gene can include the NOX2 gene also known as the CYBB gene which encodes a NOX2 protein, also known as cytochrome b-245 beta chain protein. In some embodiments, a target gene can encode a polypeptide that binds to or is associated with the NOX2 protein in vivo. Examples of such target genes include the CYBA gene which encodes a p22phox protein, the NCF1 gene which encodes neutrophil cytosolic factor 1 protein, the NCF2 gene which encodes a neutrophil cytosolic factor 2 protein, the NCF4 gene which encodes a neutrophil cytosolic factor 4 protein, the RAC1 gene which encodes a Rac1 protein, and the RAC2 gene which encodes a Rac2 protein. Accession numbers for example human genomic DNA sequences that contain certain target genes and are useful to generate targeted nucleic acids for use in a CRISPR system to reduce activity of a NOX2 protein in a cell are listed in TABLE 1.

TABLE 1

| | | Accession number for NCBI |
|---|---|---|
| Gene | Protein | reference sequence |
| NOX2 | Nox2 | NG_009065.1 |
| CYBA | p22phox | NG_007291.1 |
| NCF1 | neutrophil cytosolic factor 1 | NG_009078.2 |
| NCF2 | neutrophil cytosolic factor 2 | NG_007267.1 |
| NCF4 | neutrophil cytosolic factor 4 | NG_023400.1 |
| RAC1 | Rac1 | NG_029431.1 |
| RAC2 | Rac2 | NG_007288.1 |

Adeno-associated virus (AAV) vectors may be used to deliver CRISPRs to the cell using various construct configurations. For example, AAV may deliver Cas9 and gRNA expression cassettes on separate vectors. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit.

In some embodiments, The delivery of the CRISPR/Cas9-based system may be the transfection or electroporation of the CRISPR/Cas9-based system as a nucleic acid molecule that is expressed in the cell and delivered to the surface of the cell. The nucleic acid molecules may be electroporated using BioRad Gene Pulser Xcell or Amaxa Nucleofector IIb devices. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000. Upon delivery of the CRISPR/Cas9 system to the tissue, and thereupon the vector into the cells of the mammal, the transfected cells will express the CRISPR/Cas9-based system and/or a site-specific nuclease. In some embodiments, a modified AAV vector can be capable of delivering and expressing the site-specific nuclease in the cell of a subject. For example, the modified AAV vector may be an AAV-SASTG vector (Piacentino et al. (2012) Human Gene Therapy 23:635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy (2012) 12:139-151). In some embodiments, a cell can be modified ex vivo, and the modified cell can be delivered to a subject. In some embodiments, a modified cells may be injected or implanted into a subject, used exogenously, or developed into tissue engineered constructs.

Some embodiments include reducing the expression level of a nucleic acid encoding NOX2, or the expression level of a NOX2 protein in a cell by RNA interference and/or antisense technologies. RNA interference is an efficient process whereby double-stranded RNA (dsRNA), also referred to as siRNAs (small interfering RNAs) or ds siRNAs (double-stranded small interfering RNAs), induces the sequence-specific degradation of targeted mRNA in animal or plant cells (Hutvagner, G. et al. (2002) Curr. Opin. Genet. Dev. 12:225-232); Sharp, P. A. (2001) Genes Dev. 15:485-490). RNA interference can be triggered by various molecules, including 21-nucleotide duplexes of siRNA (Chiu, Y.-L. et al. (2002) Mol. Cell. 10:549-561. Clackson, T. et al. (1991) Nature 352:624-628.; Elbashir, S. M. et al. (2001) Nature 411:494-498), or by micro-RNAs (miRNA), functional small-hairpin RNA (shRNA), or other dsRNAs which can be expressed in vivo using DNA templates with RNA polymerase III promoters (Zheng, B. J. (2004) Antivir. Ther. 9:365-374; Paddison, P. J. et al. (2002) Genes Dev. 16:948-958; Lee, N. S. et al. (2002) Nature Biotechnol. 20:500-505; Paul, C. P. et al. (2002) Nature Biotechnol. 20:505-508; Tuschl, T. (2002) Nature Biotechnol. 20:446-448; Yu, J.-Y. et al. (2002) Proc. Natl. Acad. Sci. USA 99(9):6047-6052; McManus, M. T. et al. (2002) RNA 8:842-850; Sui, G. et al. (2002) Proc. Natl. Acad. Sci. USA 99(6):5515-5520, each of which are incorporated herein by reference in their entirety).

In some embodiments, reducing the expression level of a nucleic acid encoding NOX2 or the expression level of NOX2 protein in a cell can include contacting the cell with an isolated nucleic acid selected from the group consisting of a guide RNA (gRNA), small hairpin RNA (shRNA), a small interfering RNA (siRNA), a micro RNA (miRNA), an antisense polynucleotide, and a ribozyme. In some embodiments, the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof.

A fragment of a polynucleotide sequence can include any nucleotide fragment having, for example, at least about 5 successive nucleotides, at least about 12 successive nucleotides, at least about 15 successive nucleotides, at least about 18 successive nucleotides, or at least about 20 successive nucleotides of the sequence from which it is derived. An upper limit for a fragment can include, for example, the total number of nucleotides in a full-length sequence encoding a particular polypeptide. A fragment of a polypeptide sequence can include any polypeptide fragment having, for example, at least about 5 successive residues, at least about 12 successive residues, at least about 15 successive residues, at least about 18 successive residues, or at least about 20 successive residues of the sequence from which it is derived. An upper limit for a fragment can include, for example, the total number of residues in a full-length sequence of a particular polypeptide.

Some embodiments include reducing the expression level of a nucleic acid encoding NOX2, or the expression level of a NOX2 protein in a cell by at least about 5%, 10%, 20%, 30%, 40%, 50%, 60%, 70%, 80%, 90%, 100%, or any percentage within a range of any two of the foregoing percentages.

As used herein, “antisense polynucleotide” can include a nucleic acid that binds to a target nucleic acid, such as a RNA or DNA. An antisense polynucleotide can upregulate or downregulate expression and/or function of a target nucleic acid. An antisense polynucleotide can include any exogenous nucleic acid useful in therapeutic and/or diagnostic methods. Antisense polynucleotides can include antisense RNA or DNA molecules, micro RNA, decoy RNA molecules, siRNA, enzymatic RNA, therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

As used herein, “short hairpin RNA” (“shRNA”), also known as “small hairpin RNAs”, refers to an RNA (or RNA analog) including a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. shRNA molecules are processed to generate siRNAs. shRNAs can also include one or more bulges, such as extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In some embodiments, a shRNA can include a portion of the duplex stem is a nucleic acid sequence that is complementary (e.g., perfectly complementary or substantially complementary, e.g., anti-sense) to the NOX2 target sequence. In some embodiments, one strand of the stem portion of the shRNA is sufficiently complementary (e.g., antisense) to a target RNA (e.g., a NOX2 mRNA sequence) to mediate degradation or cleavage of said target RNA via RNA interference (RNAi). Alternatively, one strand of the stem portion of the shRNA is sufficiently complementary (e.g., antisense) to a target RNA (e.g., a NOX2 mRNA sequence) to inhibit translation of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are preferably about 15 to about 50 nucleotides in length. Preferably the two stem portions are about 18 or 19 to about 21, 22, 23, 24, 25, 30, 35, 37, 38, 39, or 40 or more nucleotides in length. In some embodiments, the length of the stem portions should be 21 nucleotides or greater. When used in mammalian cells, the length of the stem portions should be less than about 30 nucleotides to avoid provoking non-specific responses like the interferon pathway.

As used herein, the term “small interfering RNA” (“siRNA”), also referred to in the art as “short interfering RNAs”, refers to an RNA or RNA analog comprising between about 10-50 nucleotides or nucleotide analogs which is capable of directing or mediating RNA interference. Preferably, an siRNA comprises between about 15-30 nucleotides or nucleotide analogs, between about 16-25 nucleotides or nucleotide analogs, between about 18-23 nucleotides or nucleotide analogs, or between about 19-22 nucleotides or nucleotide analogs, such as 19, 20, 21 or 22 nucleotides or nucleotide analogs. The term “short” siRNA can refer to a siRNA comprising about 21 nucleotides or nucleotide analogs, for example, 19, 20, 21 or 22 nucleotides. The term “long” siRNA can refer to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, such as enzymatic processing to a short siRNA.

As used herein, “microRNA” (“miRNA”), also referred to in the art as “small temporal RNAs” (“stRNAs”), can refer to a small (10-50 nucleotide) RNA or nucleotide analogs which can be genetically encoded, such as by viral, mammalian, or plant genomes, or synthetically produced and is capable of directing or mediating RNA silencing. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing with the miRNA and most commonly results in translational inhibition or destabilization of the target mRNA.

In some embodiments, an siRNA is a duplex consisting of a sense strand and complementary antisense strand, the antisense strand having sufficient complementary to a NOX2 sequence to mediate RNAi. In some embodiments, an miRNA is optionally a duplex consisting of a 3′ strand and complementary 5′ strand, the 5′ strand having sufficient complementary to a NOX2 sequence to mediate RNAi. In some embodiments, the siRNA or miRNA molecule has a length from about 10-50 or more nucleotides, i.e., each strand comprises 10-50 nucleotides (or nucleotide analogs). In some embodiments, the siRNA or miRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. In some embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. In some embodiments, the siRNA molecule has a length from about 10-50 or more nucleotides, i.e., each strand comprises 10-50 nucleotides (or nucleotide analogs). In some embodiments, the siRNA or miRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand. siRNAs or miRNAs can be designed by using any method known in the art. The siRNAs or miRNAs provided herein can be chemically synthesized, or can be transcribed in vitro from a DNA template, or in vivo from, e.g., shRNA. The dsRNA molecules can be designed using any method known in the art.

In some embodiments, miRNAs can regulate gene expression at the post transcriptional or translational level. One common feature of miRNAs is that they are all excised from an approximately 70 nucleotides precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (See e.g., Zheng, B. J. (2004) Antivir. Ther. 9:365-374). When expressed by DNA vectors containing polymerase III promoters, micro-RNA designed hairpins can silence gene expression, such as NOX2 expression.

An example method for designing dsRNA molecules is provided in the pSUPER RNAi SYSTEM™ (OligoEngine, Seattle, Wash.). The system provides inducible expression of a siRNA in a transfected cell. To effect silencing of a specific gene, a pSUPERIOR vector is used in concert with a pair of custom oligonucleotides that include a unique 19-nt sequence derived from the mRNA transcript of the gene targeted for suppression (the “N-19 target sequence”). The N-19 target sequence corresponds to the sense strand of the pSUPER-generated siRNA, which in turn corresponds to a 19-nt sequence within the mRNA. In the mechanism of RNAi, the antisense strand of the siRNA duplex hybridizes to this region of the mRNA to mediate cleavage of the molecule. These forward and reverse oligonucleotides are annealed and cloned into the vector so that the desired siRNA duplex can be generated. The sequence of the forward oligonucleotide includes the unique N-19 target in both sense and antisense orientation, separated by a 9-nt spacer sequence. The resulting transcript of the recombinant vector is predicted to fold back on itself to form a 19-base pair stem-loop structure. The stem-loop precursor transcript is quickly cleaved in the cell to produce a functional siRNA (T. R. Brummelkamp, et al, Science 296, 550 (2002)). More example methods are provided in Taxman D. J. et al. (2006) BMC Biotechnol. 6:7; and McIntyre G. J. et al. (2006) BMC Biotechnol. 6:1, each of which is incorporated by reference in its entirety.

As used herein, “ribozyme” can include a catalytic RNA molecule that cleaves RNA in a sequence specific manner Ribozymes that cleave themselves are known as cis-acting ribozymes, while ribozymes that cleave other RNA molecules are known as trans-acting ribozymes. The term “cis-acting ribozyme sequence” as used herein refers to the sequence of an RNA molecule that has the ability to cleave the RNA molecule containing the cis-acting ribozyme sequence. A cis-acting ribozyme sequence can contain any sequence provided it has the ability to cleave the RNA molecule containing the cis-acting ribozyme sequence. For example, a cis-acting ribozyme sequence can have a sequence from a hammerhead, axhead, or hairpin ribozyme. In addition, a cis-acting ribozyme sequence can have a sequence from a hammerhead, axhead, or hairpin ribozyme that is modified to have either slow cleavage activity or enhanced cleavage activity. For example, nucleotide substitutions can be made to modify cleavage activity (Doudna and Cech, Nature, 418:222-228 (2002)). Examples of ribozyme sequences that can be used with the methods and compositions described herein include those described in U.S. Pat. Nos. 6,271,359, and 5,824,519, incorporated by reference in their entireties. One example method for preparing a ribozyme is to synthesize chemically an oligodeoxyribonucleotide with a ribozyme catalytic domain (approximately 20 nucleotides) flanked by sequences that hybridize to the target mRNA. The oligodeoxyribonucleotide is amplified by using the substrate binding sequences as primers. The amplified product is cloned into a eukaryotic expression vector. A ribozyme can be expressed in eukaryotic cells from the appropriate DNA vector. If desired, the activity of the ribozyme may be augmented by its release from the primary transcript by a second ribozyme (Ohkawa et al., Nucleic Acids Symp. Ser., 27: 15-6 (1992); Taira et al., Nucleic Acids Res., 19: 5125-30 (1991); Ventura et al., Nucleic Acids Res., 21, 3249-55 (1993).

In some embodiments, an isolated nucleic acid can include an antisense nucleic acid sequence selected such that it is complementary to the entirety of NOX2 or to a portion of NOX2. In some embodiments, a portion can refer to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, and at least about 80%, at least about 85%, at least about 90%, at least about 95%, or any portion within a range of any two of the foregoing percentages. In some embodiments, a portion can refer up to 100%. An example mRNA sequence (SEQ ID NO:01) of human NOX2 is shown in TABLE 2.

TABLE 2

| | |
|---|---|
| 1 | attggaagaa gaagcatagt atagaagaaa ggcaaacaca acacattcaa cctctgccac |
| 61 | catggggaac tgggctgtga atgaggggct ctccattttt gtcattctgg tttggctggg |
| 121 | gttgaacgtc ttcctctttg tctggtatta ccgggtttat gatattccac ctaagttctt |
| 181 | ttacacaaga aaacttcttg ggtcagcact ggcactggcc agggcccctg cagcctgcct |
| 241 | gaatttcaac tgcatgctga ttctcttgcc agtctgtcga aatctgctgt ccttcctcag |
| 301 | gggttccagt gcgtgctgct caacaagagt tcgaagacaa ctggacagga atctcacctt |
| 361 | tcataaaatg gtggcatgga tgattgcact tcactctgcg attcacacca ttgcacatct |
| 421 | atttaatgtg gaatggtgtg tgaatgcccg agtcaataat tctgatcctt attcagtagc |
| 481 | actctctgaa cttggagaca ggcaaaatga aagttatctc aattttgctc gaaagagaat |
| 541 | aaagaaccct gaaggaggcc tgtacctggc tgtgaccctg ttggcaggca tcactggagt |
| 601 | tgtcatcacg ctgtgcctca tattaattat cacttcctcc accaaaacca tccggaggtc |
| 661 | ttactttgaa gtcttttggt acacacatca tctctttgtg atcttcttca ttggccttgc |
| 721 | catccatgga gctgaacgaa ttgtacgtgg gcagaccgca gagagtttgg ctgtgcataa |
| 781 | tataacagtt tgtgaacaaa aaatctcaga atggggaaaa ataaaggaat gcccaatccc |
| 841 | tcagtttgct ggaaaccctc ctatgacttg gaaatggata gtgggtccca tgtttctgta |
| 901 | tctctgtgag aggttggtgc ggttttggcg atctcaacag aaggtggtca tcaccaaggt |
| 961 | ggtcactcac cctttcaaaa ccatcgagct acagatgaag aagaaggggt tcaaaatgga |
| 1021 | agtgggacaa tacatttttg tcaagtgccc aaaggtgtcc aagctggagt ggcacccttt |
| 1081 | tacactgaca tccgcccctg aggaagactt ctttagtatc catatccgca tcgttgggga |
| 1141 | ctggacagag gggctgttca atgcttgtgg ctgtgataag caggagtttc aagatgcgtg |
| 1201 | gaaactacct aagatagcgg ttgatgggcc ctttggcact gccagtgaag atgtgttcag |
| 1261 | ctatgaggtg gtgatgttag tgggagcagg gattggggtc acacccttcg catccattct |
| 1321 | caagtcagtc tggtacaaat attgcaataa cgccaccaat ctgaagctca aaaagatcta |
| 1381 | cttctactgg ctgtgccggg acacacatgc ctttgagtgg tttgcagatc tgctgcaact |
| 1441 | gctggagagc cagatgcagg aaaggaacaa tgccggcttc ctcagctaca acatctacct |
| 1501 | cactggctgg gatgagtctc aggccaatca ctttgctgtg caccatgatg aggagaaaga |
| 1561 | tgtgatcaca ggcctgaaac aaaagacttt gtatggacgg cccaactggg ataatgaatt |
| 1621 | caagacaatt gcaagtcaac accctaatac cagaatagga gttttcctct gtggacctga |
| 1681 | agccttggct gaaaccctga gtaaacaaag catctccaac tctgagtctg gccctcgggg |
| 1741 | agtgcatttc attttcaaca aggaaaactt ctaacttgtc tcttccatga ggaaataaat |
| 1801 | gtgggttgtg ctgccaaatg ctcaaataat gctaattgat aatataaata ccccctgctt |
| 1861 | aaaaatggac aaaaagaaac tataatgtaa tggttttccc ttaaaggaat gtcaaagatt |
| 1921 | gtttgatagt gataagttac atttatgtgg agctctatgg ttttgagagc acttttacaa |
| 1981 | acattatttc atttttttcc tctcagtaat gtcagtggaa gttagggaaa agattcttgg |
| 2041 | actcaatttt agaatcaaaa gggaaaggat caaaaggttc agtaacttcc ctaagattat |
| 2101 | gaaactgtga ccagatctag cccatcttac tccaggtttg atactctttc cacaatactg |
| 2161 | agctgcctca gaatcctcaa aatcagtttt tatattcccc aaaagaagaa ggaaaccaag |
| 2221 | gagtagctat atatttctac tttgtgtcat ttttgccatc attattatca tactgaagga |
| 2281 | aattttccag atcattagga cataatacat gttgagagtg tctcaacact tattagtgac |
| 2341 | agtattgaca tctgagcata ctccagttta ctaatacagc agggtaactg ggccagatgt |
| 2401 | tctttctaca gaagaatatt ggattgattg gagttaatgt aatactcatc atttaccact |
| 2461 | gtgcttggca gagagcggat actcaagtaa gttttgttaa atgaatgaat gaatttagaa |
| 2521 | ccacacaatg ccaagataga attaatttaa agccttaaac aaaatttatc taaagaaata |
| 2581 | acttctatta ctgtcataga ccaaaggaat ctgattctcc ctagggtcaa gaacaggcta |
| 2641 | aggatactaa ccaataggat tgcctgaagg gttctgcaca ttcttatttg aagcatgaaa |
| 2701 | aaagagggtt ggaggtggag aattaacctc ctgccatgac tctggctcat ctagtcctgc |
| 2761 | tccttgtgct ataaaataaa tgcagactaa tttcctgccc aaagtggtct tctccagcta |
| 2821 | gcccttatga atattgaact taggaattgt gacaaatatg tatctgatat ggtcatttgt |
| 2881 | tttaaataac acccacccct tattttccgt aaatacacac acaaaatgga tcgcatctgt |
| 2941 | gtgactaatg gtttattt t attatatcat catcatcatc ctaaaattaa caacccagaa |
| 3001 | acaaaaatct ctatacagag atcaaattca cactcaatag tatgttctga atatatgttc |
| 3061 | aagagagagt ctctaaatca ctgttagtgt ggccaagagc agggttttct ttttgttctt |
| 3121 | agaactgctc ccatttctgg gaactaaaac cagttttatt tgccccaccc cttggagcca |
| 3181 | caaatgttta gaactcttca acttcggtaa tgaggaagaa ggagaaagag ctgggggaag |
| 3241 | ggcagaagac tggtttagga ggaaaaggaa ataaggagaa aagagaatgg gagagtgaga |
| 3301 | gaaaataaaa aaggcaaaag ggagagagag gggaaggggg tctcatattg gtcattccct |
| 3361 | gccccagatt tcttaaagtt tgatatgtat agaatataat tgaaggaggt atacacatat |
| 3421 | tgatgttgtt ttgattatct atggtattga atcttttaaa atctggtcac aaattttgat |
| 3481 | gctgaggggg attattcaag ggactaggat gaactaaata agaactcagt tgttctttgt |
| 3541 | catactacta ttcctttcgt ctcccagaat cctcagggca ctgagggtag gtctgacaaa |
| 3601 | taaggcctgc tgtgcgaata tagcctttct gaaatgtacc aggatggttt ctgcttagag |
| 3661 | acacttaggt ccagcctgtt cacactgcac ctcaggtatc aattcatcta ttcaacagat |
| 3721 | atttattgtg ttattactat gagtcaggct ctgtttattg tttcaattct ttacaccaaa |
| 3781 | gtatgaactg gagagggtac ctcagttata aggagtctga gaatattggc cctttctaac |
| 3841 | ctatgtgcat aattaaaacc agcttcattt gttgctccga gagtgtttct ccaaggtttt |
| 3901 | ctatcttcaa aaccaactaa gttatgaaag tagagagatc tgccctgtgt tatccagtta |
| 3961 | tgagataaaa aatgaatata agagtgcttg tcattataaa agtttccttt tttattctct |
| 4021 | caagccacca gctgccagcc accagcagcc agctgccagc ctagcttttt tttttttttt |
| 4081 | ttttttttag cacttagtat ttagcattta ttaacaggta ctctaagaat gatgaagcat |
| 4141 | tgtttttaat cttaagacta tgaaggtttt tcttagttct tctgcttttg caattgtgtt |
| 4201 | tgtgaaattt gaatacttgc aggctttgta tgtgaataat tctagcgggg gacctgggag |
| 4261 | ataattccta cggggaattc ttaaaactgt gctcaactat taaaatgaat gagctttcaa |
| 4321 | aaaaaaaaaa aaaaaaaaaa aaaaaaaaaa aaa |

(SEQ ID NO: 01)
DEFINITION: Homo sapiens cytochrome b-245 beta chain (CYBB), mRNA
ACCESSION: NM_000397
VERSION: NM_000397.3

In some embodiments, an antisense oligonucleotide can have a length of at least about 5 nucleotides, at least about 7 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides. An antisense nucleic acid of disclosed herein can be constructed using chemical synthesis and enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids, such as phosphorothioate derivatives and acridine substituted nucleotides can be used. The antisense nucleic acid also can be produced biologically using an expression vector into which a nucleic acid has been subcloned in an antisense orientation, namely, RNA transcribed from the inserted nucleic acid will be of an antisense orientation to a target nucleic acid of interest. The antisense nucleic acid molecules can be administered to a subject, such as systemically or locally by direct injection at a tissue site, or generated in situ such that they hybridize with or bind to cellular mRNA and/or genomic DNA encoding NOX2 to thereby inhibit its expression. Alternatively, antisense nucleic acid molecules can be modified to target particular cells and then administered systemically. For systemic administration, antisense molecules can be modified such that they specifically bind to receptors or antigens expressed on a selected cell surface, e.g., by linking the antisense nucleic acid molecules to peptides or antibodies that bind to particular cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In some embodiments, antisense oligonucleotide include a-anomeric nucleic acid molecules. An α-anomeric nucleic acid molecule forms specific double-stranded hybrids with complementary RNA in which, contrary to the usual beta-units, the strands run parallel to each other (Gaultier, C. et al. (1987) Nucleic Acids. Res. 15:6625-6641). The antisense nucleic acid molecule can also comprise a 2′-o-methylribonucleotide, or a chimeric RNA-DNA analogue (Inoue, H. et al. (1987) Nucleic Acids Res. 15:6131-6148; Inoue, H. et al. (1987a) FEBS Lett. 215:327-330).

In some embodiments, an isolated nucleic acid can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, such as the methods of Lambert, G. et al. (2001) Drug Deliv. Rev. 47(1): 99-112 (describes nucleic acids loaded to polyalkylcyanoacrylate (PACA) nanoparticles); Fattal et al. (1998) J. Control Release 53(1-3): 137-43 (describes nucleic acids bound to nanoparticles); Schwab et al. (1994) Ann. Oncol. 5 Suppl. 4:55-58 (describes nucleic acids linked to intercalating agents, hydrophobic groups, polycations or PACA nanoparticles); and Godard, G. et al. (1995) Eur. J. Biochem. 232(2):404-10 (describes nucleic acids linked to nanoparticles). Because RNAi is believed to progress via at least one single stranded RNA intermediate, the skilled artisan will appreciate that ss-siRNAs (e.g., the antisense strand of a ds-siRNA) can also be designed as described herein and utilized according to the claimed methodologies.

Some embodiments reducing the expression level of a nucleic acid encoding NOX2, or the expression level of a NOX2 protein in a cell can include delivering an isolated nucleic acid, such as an siRNA to a cell by methods known in the art, including cationic liposome transfection and electroporation. In some embodiments, an siRNA can show short term persistence of a silencing effect which may be beneficial in certain embodiments. To obtain longer term suppression of expression for targeted genes, such as NOX2, and to facilitate delivery under certain circumstances, one or more siRNA duplexes, such as a ds siRNA, can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, T. (2002) Nature Biotechnol. 20:446-448) capable of expressing functional double-stranded siRNAs; (Lee, N. S. et al. (2002) Nature Biotechnol. 20:500-505; Miyagishi, M. and Taira, K. (2002) Nature Biotechnol. 20:497-500; Paul, C. P. et al. (2002) Nature Biotechnol. 20:505-508; Yu, J.-Y. et al. (2002) Proc. Natl. Acad. Sci. USA 99(9):6047-6052; Sui, G. et al. (2002) Proc. Natl. Acad. Sci. USA 99(6):5515-5520). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase (Jacque J.-M. et al. (2002) Nature 418:435-438). A single construct may contain multiple sequences coding for siRNAs, such as multiple regions of the NOX2 gene, such as a nucleic acid encoding the NOX2 mRNA, and can be driven, for example, by separate Pol III promoter sites.

Some embodiments reducing the expression level of a nucleic acid encoding NOX2, or the expression level of a NOX2 protein in a cell can include viral-mediated delivery of certain isolated nucleic acids to a cell. In some such embodiments, specific silencing of targeted genes through expression of certain nucleic acids, such as an siRNA by generating recombinant adenoviruses harboring siRNA under RNA Pol II promoter transcription control (Xia et al. (2002) Nature Biotechnol. 20(10):1006-10). Injection of recombinant adenovirus vectors into transgenic mice expressing the target genes of the siRNA results in in vivo reduction of target gene expression. In adult mice, efficient delivery of siRNA can be accomplished by the “high-pressure” delivery technique, a rapid injection (within 5 seconds) of a large volume of siRNA containing solution into animal via the tail vein (Lewis, D. L. (2002) Nature Genetics 32:107-108). Nanoparticles, liposomes and other cationic lipid molecules can also be used to deliver siRNA into animals. A gel-based agarose/liposome/siRNA formulation is also available (Jiamg, M. et al. (2004) Oligonucleotides 14(4):239-48).

### Methods of Reducing Levels of ROS

Some embodiments of the methods and compositions provided herein include decreasing the level of ROS production in a population of cells, such as a population comprising metastatic tumor cells. In some such embodiments, decreasing the level of ROS production in the population of cells can include reducing the activity of NOX2 in a cell of the population. In some embodiments, a metastatic tumor comprises the metastatic tumor cells. In some embodiments, the cell is a myeloid cell. In some such embodiments, the level of ROS production in the population of cells in which the activity of NOX2 in a cell of the population has been reduced is decreased compared to the level of ROS production in a population of cells in which the activity of NOX2 in a cell has not been reduced. In some embodiments, the level of production of ROS in the population of cells can be decreased by at least about 5%, 10%, 20%, 30%, 40%, 50%, 60%, 70%, 80%, 90%, 100%, or any percentage in a range between any two of the foregoing percentages.

### Methods of Increasing NK Cells

Some embodiments of the methods and compositions provided herein include increasing the level of NK cells in a lung of a subject by reducing the activity of NOX2 in a cell of a subject. In some embodiments, the lung comprises a metastatic tumor. In some embodiments the cell is a myeloid cell. In some such embodiments, the level of NK cells in a lung of a subject in which the activity of NOX2 in a cell of a subject has been reduced is decreased compared to the level of NK cells in a subject in which the activity of NOX2 in a cell of the subject has not been reduced. In some embodiments, the level of NK cells in a lung of a subject can be increased by at least about 5%, 10%, 20%, 30%, 40%, 50%, 60%, 70%, 80%, 90%, 100%, 150%, 200%, 300%, 500%, or any percentage in a range between any two of the foregoing percentages.

### Combination Therapies

Some embodiments of the methods and compositions provided herein include contacting a cell and/or administering to a subject a NOX2 inhibitor or isolated nucleic acid in combination with an additional therapeutic agent. As used herein, administering in combination can include administering two or more agents to a subject, such as a NOX2 inhibitor or isolated nucleic acid and an additional therapeutic agent, such that the two or more agents may be found in the subject's bloodstream at the same time, regardless of when or how they are actually administered. In some embodiments, the agents are administered simultaneously. In some such embodiments, administration in combination is accomplished by combining the agents in a single dosage form. When combining the agents in a single dosage form, they may be physically mixed, such as by co-dissolution or dry mixing, or may form an adduct or be covalently linked such that they split into the two or more active ingredients upon administration to the subject. In some embodiments, the agents are administered sequentially. In some embodiments, the agents are administered through the same route, such as orally. In some embodiments, the agents are administered through different routes, such as one being administered orally and another being administered i.v.

In some embodiments, the additional therapeutic agent can include a NK cell activating agent. Examples of such NK activating agents include IL-15, IFN-γ, IL-12, IL-18, IL-2, and CCL5. In some embodiments, the additional therapeutic agent is IL-15. In some embodiments, the additional therapeutic agent is IFN-γ.

In some embodiments, the additional therapeutic agent can include a chemotherapeutic agent. In some embodiments, the chemotherapeutic agent is a cell cycle inhibitor. As used herein, “cell cycle inhibitor” can include a chemotherapeutic agent that inhibits or prevents the division and/or replication of cells. In some embodiments, “cell cycle inhibitor” can include a chemotherapeutic agent such as Doxorubicin, Melphlan, Roscovitine, Mitomycin C, Hydroxyurea, 50Fluorouracil, Cisplatin, Ara-C, Etoposide, Gemcitabine, Bortezomib, Sunitinib, Sorafenib, Sodium Valproate, a HDAC Inhibitors, or Dacarbazine. More examples of additional chemotherapeutic agents include HDAC inhibitors such as FR01228, Trichostatin A, SAHA and PDX101. In some embodiments, the cell cycle inhibitor is a DNA synthesis inhibitor. As used herein, “DNA synthesis inhibitor” can include a chemotherapeutic agent that inhibits or prevents the synthesis of DNA by a cancer cell. Examples of DNA synthesis inhibitors include AraC (cytarabine), 6-mercaptopurine, 6-thioguanine, 5-fluorouracil, capecitabine, floxuridine, gemcitabine, decitabine, vidaza, fludarabine, nelarabine, cladribine, clofarabine, pentostatin, thiarabine, troxacitabine, sapacitabine or forodesine. More examples of additional chemotherapeutic agents include FLT3 inhibitors such as Semexanib (SU5416), Sunitinib (SU11248), Midostaurin (PKC412), Lestautinib (CEP-701), Tandutinib (MLN518), CHIR-258, Sorafenib (BAY-43-9006) and KW-2449. More examples of additional chemotherapeutic agents include farnesyltransferase inhibitors such as tipifarnib (R115777, Zarnestra), lonafarnib (SCH66336, Sarasar™) and BMS-214662. More examples of additional chemotherapeutic agents include topoisomerase II inhibitors such as the epipodophyllotoxins etoposide and teniposide, and the anthracyclines doxorubicin and 4-epi-doxorubicin. More examples of additional chemotherapeutic agents include P-glycoprotein modulators such as zosuquidar trihydrochloride (Z.3HCL), vanadate, and/or verapamil. More examples of additional chemotherapeutic agents include hypomethylating agents such as 5-aza-cytidine and/or 2′ deoxyazacitidine.

### Pharmaceutical Compositions and Formulations

Some embodiments of the methods and compositions provided herein include pharmaceutical compositions, and administration of such compositions. In some embodiments, a pharmaceutical composition can include a NOX2 inhibitor, such as a therapeutically effective amount of a NOX2 inhibitor. In some embodiments, a pharmaceutical composition can include a NOX2 inhibitor and a pharmaceutically acceptable excipient. As used herein, a “pharmaceutically acceptable” can include a carrier, diluent or excipient that does not abrogate the biological activity and properties of a NOX2 inhibitor. In some embodiments, pharmaceutical composition can include a NOX2 inhibitor and an additional therapeutic agent. Standard pharmaceutical formulation techniques can be used, such as those disclosed in Remington's The Science and Practice of Pharmacy, 21st Ed., Lippincott Williams & Wilkins (2005), incorporated by reference in its entirety.

In some embodiments, a pharmaceutical composition can be administered to a subject by any of the accepted modes of administration for agents that serve similar utilities including, but not limited to, orally, subcutaneously, intravenously, intranasally, topically, transdermally, intraperitoneally, intramuscularly, intrapulmonary, vaginally, rectally, or intraocularly.

In some embodiments, a pharmaceutical composition comprising a NOX2 inhibitor can be administered at a therapeutically effective dosage, such as a dosage sufficient to provide treatment for a disorder. The amount of active compound administered will, of course, be dependent on the subject and disease state being treated, the severity of the disorder, the manner and schedule of administration and the judgment of the prescribing physician. The actual dose of the active compounds, such as NOX2 inhibitors depends on the specific compound, and on the condition to be treated; the selection of the appropriate dose is well within the knowledge of the skilled artisan.

In some embodiments, the pharmaceutical composition is administered subcutaneously. Solutions of an active compound, such as a NOX2 inhibitor, as a free acid or a pharmaceutically-acceptable salt may be administered in water with or without a surfactant such as hydroxypropyl cellulose. Dispersions are also contemplated such as those utilizing glycerol, liquid polyethylene glycols and mixtures thereof and oils. Antimicrobial compounds may also be added to the preparations. Injectable preparations may include sterile aqueous solutions or dispersions and powders which may be diluted or suspended in a sterile environment prior to use. Carriers such as solvents dispersion media containing, e.g., water, ethanol polyols, vegetable oils and the like, may also be added. Coatings such as lecithin and surfactants may be utilized to maintain the proper fluidity of the composition. Isotonic agents such as sugars or sodium chloride may also be added as well as products intended for the delay of absorption of the active compounds such as aluminum monostearate and gelatin. Sterile injectable solutions are prepared as is known in the art and filtered prior to storage and/or administration. Sterile powders may be vacuum dried freeze dried from a solution or suspension containing them. In some embodiments, the pharmaceutical compositions are administered by intravenous, intra-arterial, or intra-muscular injection of a liquid preparation. Suitable liquid formulations include solutions, suspensions, dispersions, emulsions, oils and the like. In some embodiments, the pharmaceutical compositions are administered intravenously and are thus formulated in a form suitable for intravenous administration. In some embodiments, the pharmaceutical compositions are administered intra-arterially and are thus formulated in a form suitable for intra-arterial administration. In some embodiments, the pharmaceutical compositions are administered intra-muscularly and are thus formulated in a form suitable for intra-muscular administration.

Proper formulation is dependent upon the route of administration selected. For injection, the agents of the compounds may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, comprising lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds, such as NOX2 inhibitors, for use according to the present disclosure may be conveniently delivered in the form of an aerosol spray presentation from pressurized packs or a nebulizer, with the use of a suitable propellant, such as carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds, such as NOX2 inhibitors, may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds, such as NOX2 inhibitors, in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

In addition to the formulations described herein, the compounds, such as NOX2 inhibitors, may also be formulated as a depot preparation. Such long-acting formulations may be administered by implantation, such as subcutaneously or intramuscularly, or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

In some embodiments, delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds, such as NOX2 inhibitors, may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutically acceptable formulations can contain a compound, or a salt or solvate thereof, in an amount of about 50 mg, about 100 mg, about 150 mg, about 200 mg, about 250 mg, about 300 mg, about 350 mg, about 400 mg, about 450 mg, or about 500 mg. Additionally, the pharmaceutically acceptable formulations may contain a compound such as NOX2 inhibitor, or a salt or solvate thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

### Kits

Some embodiments of the methods and compositions provided herein include kits comprising a NOX2 inhibitor and/or an isolated nucleic acid, wherein the isolated nucleic acid reduces the expression level of a nucleic acid encoding NOX2 or the expression level of NOX2 protein in a cell. In some embodiments, the NOX2 inhibitor can include histamine dihydrochloride (HDC), histamine, N-methyl-histamine, 4-methyl-histamine, histamine phosphate, histamine diphosphate, GSK2795039, apocynin, GKT136901, GKT137831, ML171, VAS2870, VAS3947, celastrol, ebselen, perhexiline, grindelic acid, NOX2ds-tat, NOXA1ds, fulvene-5, ACD 084, NSC23766, CAS 1177865-17-6, and CAS 1090893-12-1, and shionogi. In some embodiments, the NOX2 inhibitor is HDC. In some embodiments, the isolated nucleic acid can include a guide RNA (gRNA), a small hairpin RNA (shRNA), a small interfering RNA (siRNA), a micro RNA (miRNA), an antisense polynucleotide, and a ribozyme. In some embodiments, the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof.

In some embodiments, a kit can include an additional therapeutic agent. In some embodiments, the additional therapeutic agent is a NK cell activating agent. In some embodiments, the NK cell activating agent can include IL-15, IFN-γ, IL-12, IL-18, IL-2, and CCL5. In some embodiments, the additional therapeutic agent is IL-15. In some embodiments, the additional therapeutic agent is IFN-γ. In some embodiments, the additional therapeutic agent can include a chemotherapeutic agent.

In some embodiments, a kit can include reagents to generate the modified cell. In some such embodiments, a kit can include reagents useful for use with a CRISPR system. In some embodiments, reagents can include a modified AAV vector and a nucleotide sequence encoding a site-specific nuclease. The site-specific nuclease may include a ZFN, a TALEN, or CRISPR/Cas9-based system that specifically binds and cleaves a modified target gene, such as a modified NOX2 gene. The site-specific nuclease may be included in the kit to specifically bind and target a particular region in the endogenous target gene, such as a NOX2 target gene. The kit may further include donor DNA, a gRNA, or a transgene. In some embodiments, a kit can include a Cas9 protein or Cas9 fusion protein, a nucleotide sequence encoding a Cas9 protein or Cas9 fusion protein, and/or at least one gRNA. The CRISPR/Cas9-based system may be included in the kit to specifically bind and target a particular target region upstream, within or downstream of the coding region of the target gene, such as a NOX2 gene. For example, a CRISPR/Cas9-based system may be specific for a promoter region of a target gene or a CRISPR/Cas9-based system may be specific for the coding region.

### Indications

Some embodiments of the methods and compositions provided herein include preventing, treating or ameliorating a subject having a disorder, such as preventing metastasis of a primary tumor, and treating or ameliorating a metastatic tumor. In some embodiments, the primary or metastatic tumor can include a melanoma, a bladder cancer, a breast cancer, a pancreatic cancer, a colorectal cancer, a renal cancer, a prostate cancer, a stomach cancer, a thyroid cancer, a uterine cancer, and an ovarian cancer. In some embodiments, the disorder can include a melanoma. In some embodiments, the disorder includes a primary or metastatic melanoma. Examples of melanoma include lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, and vaginal melanoma. In some embodiments, the disorder can include primary uveal melanoma. In some embodiments, the metastatic tumor is located at a site selected from the group consisting of lung, liver, brain, peritoneum, adrenal gland, skin, muscle, vagina, and bone. In some embodiments, the primary or metastatic tumor is located in a lung. In certain embodiments, the metastatic tumor comprises a melanoma located in a lung.

### EXAMPLES

### Inhibition of NOX2 Reduces Hematogenous Melanoma Metastasis

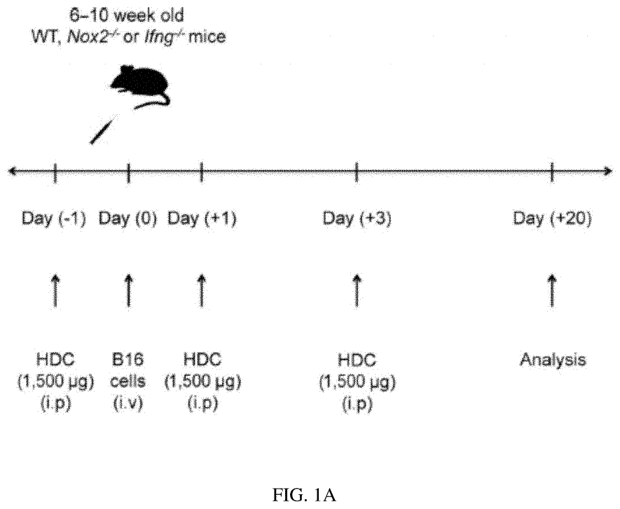

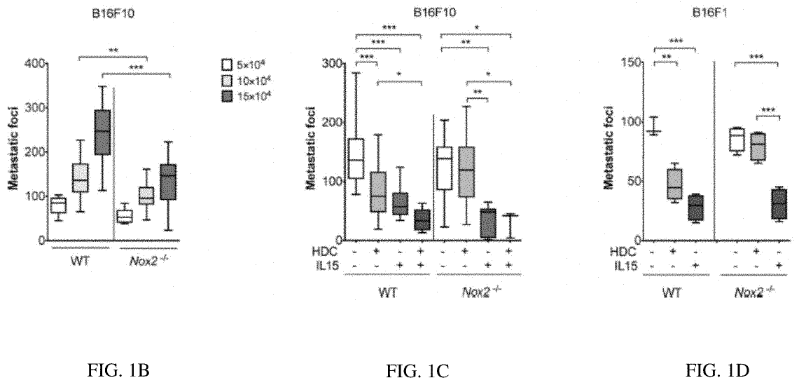

To elucidate the role of NOX2-derived ROS in murine melanoma metastasis, genetically modified mice were used that lack the myeloid gp91^{phox} subunit NOX2 and thus a functional ROS-producing NOX2 in myeloid cells (Nox2-KO mice). FIG. 1A depicts an experimental design. Over a range of amounts of intravenous (i.v.) inoculated B16F10 cells, it was observed that the establishment of melanoma metastases was less pronounced in lungs of Nox2-KO mice compared with WT B6 mice (FIG. 1B). FIG. 1B depicts the number of metastatic foci formed in lungs of WT and Nox2-KO (Nox2^{-/-}) mice at 3 weeks after i.v. inoculation of 50,000, 100,000, or 150,000 B16F10 cells.

Effects of HDC, a NOX2-inhibitor were evaluated on melanoma metastasis in WT and Nox2-KO mice. These experiments were performed using loads of injected B16F10 cells that produced comparable numbers of metastases in WT and Nox2-KO animals; that is, 100,000 B16F10 cells for WT mice and 150,000 cells for Nox2-KO mice. Systemic treatment of mice with HDC (1,500 μg/mouse intraperitoneal, i.p.) during the initial phase of melanoma engraftment (days: −1, 1, and 3 after tumor cell inoculation) decreased the number of lung metastases in WT mice. These effects were not observed in Nox2-KO mice (FIG. 1C). The NK cell-activating cytokine IL15 (24; 0.04 μg/mouse on days −1, 1, and 3) exerted antimetastatic activity in vivo in WT and Nox2-KO mice (FIG. 1C). Combined treatment with HDC and IL15 additively reduced B16F10 metastasis in WT mice but not in Nox2-KO mice (FIG. 1C). Combined treatment with IL15 and HDC was significantly more effective than IL15 alone to reduce metastasis formation in WT mice, when analyzed by t test, P=0.01 (up to five independent experiments). In the experiments shown in FIG. 1C, n=15 for all groups of WT mice; n=8 for control, HDC and IL15 groups of Nox2^{-/-} mice and n=3 for HDC+IL15.

Experiments using the B16F1 strain of melanoma cells confirmed the reduced level of metastasis in Nox2-KO mice and the NOX2-dependent, antimetastatic effect of HDC in vivo (FIG. 1D). FIG. 1D depicts the results from lung metastasis formation by the B16F1 melanoma cell line. This cell line is less metastatic compared with B16F10 and, therefore, 200,000 B16F1 cells were injected into WT mice and 300,000 cells into Nox2^{-/-} mice. The number of metastatic foci in lungs of WT and Nox2-KO mice after systemic treatment with HDC or IL15 was determined after 3 weeks (n=4 for all groups except n=3 for control group of WT mice). The results shown in FIGS. 1C-D were evaluated by repeated measures for analysis of variance (ANOVA). Nonsignificant values: n.s., P>0.05; *, P≤0.05; **, P≤0.01; ***, P≤0.001.

### HDC Targets ROS Formation In Vitro and In Vivo

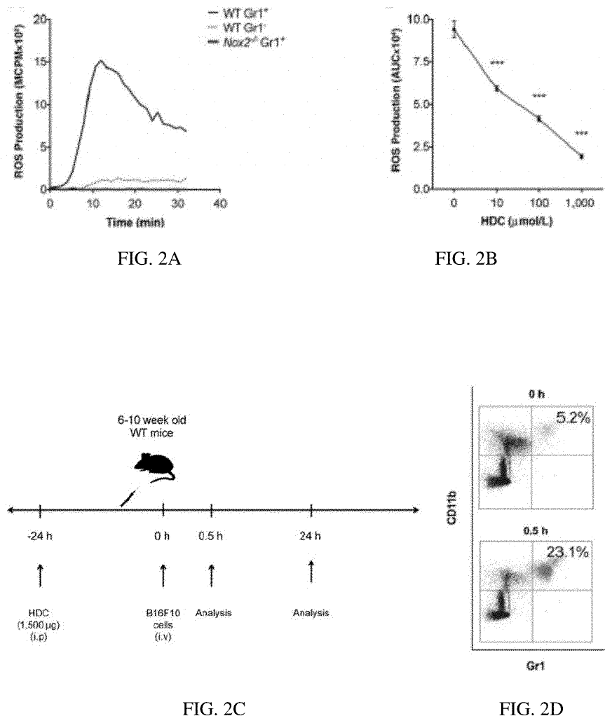

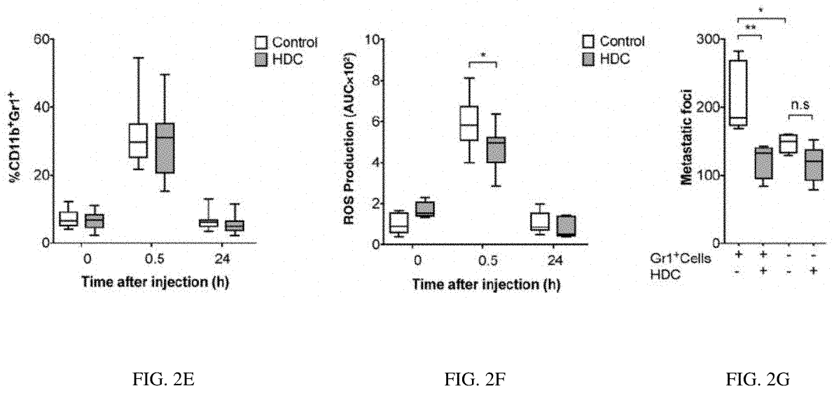

CD11b⁺Gr1⁺ myeloid cells express NOX2 and constitute the principal source of extracellular ROS in blood and tissue (26, 27). Accordingly, CD11b⁺Gr1⁺ cells isolated from the lungs of naïve WT mice, but not from Nox2-KO mice, produced extracellular ROS upon stimulation, whereas the Gr1⁻ fraction of lung cells produced minute extracellular ROS (FIG. 2A). ROS formation from WT lung cells was dose-dependently suppressed by HDC in vitro (FIG. 2B).

In experiments designed to assess the dynamics of ROS-producing myeloid cells in lungs after B16F10 cell inoculation (FIG. 2C), a pronounced and transient influx of CD11b⁺Gr1⁺ myeloid cells into lungs at 30 minutes after i.v. inoculation of tumor cells was observed (FIG. 2D and FIG. 2E). Systemic treatment with HDC prior to melanoma cell inoculation did not alter the degree of influx of myeloid cells into lungs (FIG. 2E) but reduced the ROS formed ex vivo in lung cell suspensions (FIG. 2F). To further clarify the impact of CD11b⁺Gr1⁺ cells on melanoma metastasis, Gr1⁺ cells were depleted from WT mice before treatment of mice with HDC and i.v. inoculation of B16F10 cells. The extent of lung metastasis was reduced in the absence of Gr1⁺ cells. Systemic treatment with HDC did not affect metastasis in Gr1⁺-depleted mice (FIG. 2G).

### Role of NK Cells for Melanoma Metastasis in WT and Nox2-KO Mice

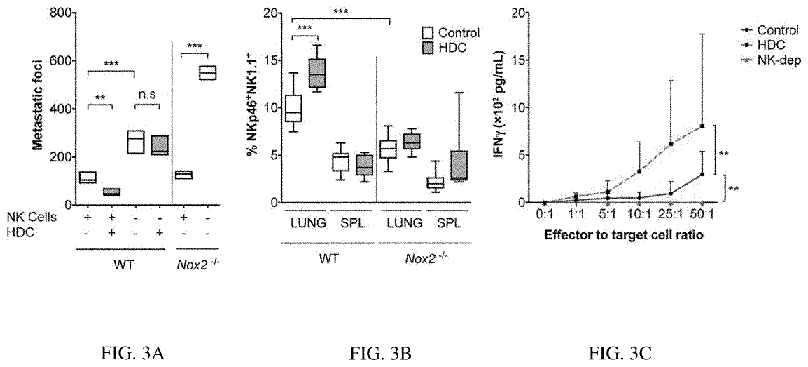

NK-cell function can limit lung metastasis in experimental models of murine melanoma. To elucidate a role of NK cells in the context of NOX2 inhibition, WT and Nox2-KO mice were depleted of NK cells by anti-NK1.1 antibody treatment prior to melanoma cell inoculation. NK-cell depletion more than doubled metastasis formation in WT and Nox2-KO mice. HDC did not inhibit melanoma metastasis in animals depleted of NK cells (FIG. 3A). FIG. 3A depicts the effects of systemic treatment with HDC on B16F10 metastasis formation in WT and Nox2^{-/-} animals depleted of NK cells (n=7 for untreated WT mice with and without NK cells (two independent experiments); n=3 for HDC-treated WT mice with and without NK cells; n=4 for each group of Nox2^{-/-} mice, one way ANOVA).

In experiments designed to clarify whether the reduced ROS levels in lungs following administration of HDC translated into altered NK-cell function at the site of tumor expansion, it was observed that treatment of mice with HDC entailed increased NK-cell counts in lungs, but not in spleen, at 3 weeks after tumor cell inoculation (FIG. 3B). FIG. 3B depicts the effects of systemic treatment with HDC on NK-cell numbers in lungs and spleens of WT and Nox-KO (Nox2^{-/-}) mice at 3 weeks after tumor cell inoculation. The percentage of NK cells out of live CD45⁺ cells was determined by flow cytometry (WT mice n=9-11; Nox2^{-/-} mice n=9-13, t test; three independent experiments). Unexpectedly, fewer NK cells in lungs and spleens of Nox2-KO mice than in WT animals were detected (FIG. 3B). Also, as shown in FIG. 3A, the degree of metastasis was strikingly enhanced in NK cell-depleted Nox2-KO mice, which may point toward the possibility of increased functionality of NK cells in the absence of NOX2.

### NOX2 Inhibition Enhances the Capacity of Lung NK Cells to Produce IFNγ

As the antimetastatic functions of NK cells in the B16 model reportedly rely on the formation of IFNγ, the IFNγ production of pulmonary NK cells from Nox2-KO and WT mice was assessed. Lung cells were isolated 30 minutes after B16F10 cell inoculation and IFNγ production was then assessed upon coculture of lung cells with B16F10 cells in vitro. Only minor amounts of IFNγ (<25 μg/mL) were detected when lung cells or B16 cells were cultured alone. Also, minute levels (<10 μg/mL) of IFNγ were produced in cocultures of lung cells and B16 cells after the depletion of NK cells in vivo using anti-NK1.1, thus supporting that the IFNγ produced in these cell cultures was contributed by NK cells (FIG. 3C). It was further observed that lung NK cells from Nox2-KO mice produced significantly higher amount of IFNγ ex vivo at a lung cell to melanoma cell ratio of 50:1 compared with lung NK cells from WT mice (WT vs. Nox2-KO lungs; 297±81 vs. 749±27 μg/mL, respectively; P=0.004, t test). A similar experimental design was adopted to assess the impact of pharmacologic NOX2 inhibition by HDC on the formation of IFNγ in lungs. Lung cells were isolated from HDC-treated or control WT mice at 30 minutes after B16F10 cell inoculation. When lung cells were cocultured with the B16 cells, higher concentrations of IFNγ were produced ex vivo by lung NK cells isolated from mice treated with HDC in vivo (FIG. 3C).

### Role of IFNγ in NOX2-Mediated Control of Melanoma Metastasis

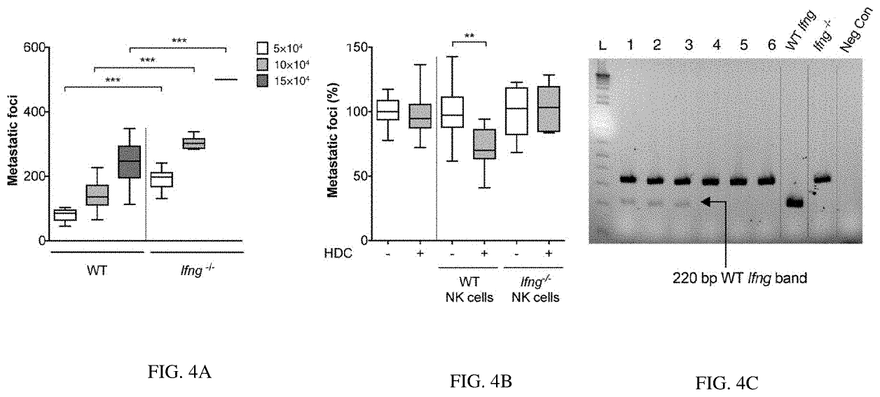

The capacity of B16F10 cells to form metastases in Ifng-KO versus WT mice was assessed. Melanoma metastasis was enhanced in IFNγ-deficient mice (FIG. 4A). Systemic treatment with HDC did not reduce melanoma metastasis in Ifng-KO mice (FIG. 4B). The adoptive transfer of IFNγ-producing WT NK cells, but not the transfer of Ifng-KO NK cells, to Ifng-KO mice significantly restored the antimetastatic efficacy of HDC (FIG. 4B). Presence of cells with Ifng^{+/+} genotype in blood of Ifng-KO mice was confirmed by PCR at 2 days after the adoptive transfer of WT NK cells (FIG. 4C).

Embodiments described herein include genetic inhibition of NOX2, which mediates oxidative stress by generating ROS from myeloid cells, reduced the capacity of two strains of murine melanoma cells (B16F1 and B16F10) to form lung metastases after i.v. inoculation, apparently by facilitating NK cell-mediated clearance of malignant cells. Also, treatment of mice with the NOX2 inhibitor HDC reduced melanoma metastasis in WT but not in Nox2-KO mice. The results show that HDC reduces the subcutaneous growth of EL-4 thymoma tumors in WT but not in Nox2-KO mice, thus, underscoring that the antineoplastic efficacy of HDC depends on the availability of NOX2.

Some embodiments described herein show that the establishment of melanoma metastases was associated with a rapid and transient accumulation of ROS-forming CD11b⁺Gr1⁺ myeloid cells in the lung parenchyma and that the ROS-forming capacity of infiltrating myeloid cells ex vivo was suppressed by the in vivo administration of HDC. Pharmacologic inhibition of NOX2 also entailed increased numbers of lung NK cells in tumor-bearing mice. The availability of IFNγ was a component for NK cell-mediated clearance of B16 melanoma cells from lungs. Furthermore, the antimetastatic effect of HDC was absent in Ifng-KO mice but could be reconstituted by the adoptive transfer of Ifng^{+/+} NK cells. Collectively, these results imply that the antimetastatic properties of HDC rely on the availability of NK cell-derived IFNγ. It was observed that despite the more efficient NK cell-mediated clearance of melanoma cells in Nox2-KO, rather than in WT mice, higher counts of NK cells were detected in the lung parenchyma of WT mice. This finding implies that NK cells were more efficient effector cells on a per cell basis in Nox2-KO mice. Consistently, pulmonary NK cells from Nox2-KO mice were observed to show enhanced formation of IFNγ ex vivo.

From these results, it was contemplated, without being bound by any particular theory, that NOX2-derived ROS produced by myeloid cells may exert oxidative stress with ensuing reduction of NK cell-mediated clearance of melanoma cells and aggravation of metastasis. In agreement with these findings, the subcutaneous growth of murine melanoma and lung carcinoma was reduced in Nox2-KO mice. Cancer cells, thus, display elevated ROS concentrations due to enhanced metabolism and mutations that trigger oxidative processes. The increased ROS may promote mutagenesis and may also render tumor cells more prone to expand and produce distant metastases. In agreement, overexpression of the antioxidant SOD3 inhibits murine breast cancer cell metastasis, and the ROS scavenger N-acetyl-cysteine reduces the tumorigenicity of murine melanoma cells.

High endogenous ROS concentrations in malignant cells can also render these cells more vulnerable to further stresses. Hence, anticancer therapies that trigger a further increase in ROS formation may induce cell death in cancer cells compared with their non-malignant counterparts. In addition, ROS can limit malignant growth by triggering activation of p53, whereas antioxidants enhanced tumor progression in a p53-dependent manner Antioxidants can enhance lymph node metastasis in a model of genetically related melanoma. In immunodeficient NOD-SCID-Il2rg^{-/-} mice, oxidative stress reduces the ability of primary melanoma cells to metastasize, whereas treatment with antioxidants enhanced metastasis.

It was contemplated, without being bound by any particular theory, that at least two mechanisms of relevance to melanoma metastasis and ROS-mediated oxidant stress were operable in immunocompetent mice, that is, direct effects of ROS on tumor cells that may either inhibit or enhance melanoma cell expansion, and oxidant-induced immunosuppression that may promote tumor growth and metastasis. The relative significance of these partly opposing mechanisms may relate to the sensitivity of melanoma cells to the growth-promoting or toxic effects of ROS as well as to the sensitivity of melanoma cells to immune-mediated clearance. This view may explain that the in vivo administration of ROS-scavenging antioxidants such as N-acetylcysteine promotes as well as prevents murine melanoma metastasis.

The source of ROS may be critical for its capacity to promote or inhibit tumor progression. In a model described herein, only ROS derived from NOX2-sufficient myeloid cells were targeted. In contrast, ROS scavengers such as N-acetyl-cysteine may also neutralize ROS generated from other sources, including those formed in mitochondria during cell respiration. The notion that NOX2+ myeloid cells may facilitate melanoma metastasis is supported by the findings that neutrophil infiltration of human primary melanomas heralds early metastatic spread and that the exposure of murine cutaneous melanomas to UV light or chemical carcinogens triggers neutrophil-dependent inflammation that promotes metastasis. Indeed, the adoptive transfer of CD11b⁺Ly6G⁺ neutrophilic granulocytes enhances the formation of lung metastases after i.v. inoculation of murine carcinoma cells. The effect was secondary to granulocyte-induced inhibition of NK-cell function. Neutrophil secretion of IL1β and matrix metalloproteinases contributed to tumor cell extravasation but the detailed mechanism of NK-cell inhibition was not defined.

Results provided herein show that the release of NOX2-derived ROS from these cells can constitute a mechanism of NK-cell inhibition during metastasis of relevance to these previous reports, and that target NOX2-derived ROS can facilitate NK cell-mediated clearance of metastatic cells. It is further supported by the results of the present study showing that the depletion of Gr1+ cells reduced melanoma metastasis, and that NOX2 inhibition using HDC did not affect metastasis in Gr1+-depleted animals. Additionally, the finding that IL15, an NK-cell-activating cytokine, improved the antimetastatic efficacy of pharmacologic NOX2 inhibition in WT animals supports a combinatorial immunotherapy to reduce metastasis formation.

In summary, results described herein suggest that NOX2 function affects NK cell-mediated control of murine melanoma metastasis. Pharmacologic inhibition of NOX2, alone or combined with immunostimulatory strategies can be useful in preventing melanoma metastasis.

### Materials and Methods

### Culture of Cell Lines

B16F1 and B16F10 murine melanoma cells were obtained in 2013 from the Cell Culture Laboratory at the Department of Virology, University of Gothenburg, where cells were authenticated by melanotic morphology and checked for absence of mycoplasma using PCR before freezing aliquots. Each aliquot was thawed and cultured for no more than 1 week for each experiment. Cells were cultured in Iscoves' medium containing 10% FCS (Sigma-Aldrich), 2 mmol/L L-glutamine, 1 mmol/L sodium pyruvate, 100 U/mL penicillin, and 100 mg/mL streptomycin at 37° C., 5% CO₂ for 1 week before inoculation into mice.

### Induction of Lung Metastasis in Nox2-KO and Ifng-KO Mice

C57BL/6 mice were obtained from The Charles River Laboratories. B6.129S6-Cybb^{tm1Din}(Nox2^{-/-} or Nox2-KO) mice that lack the myeloid gp91^{phox} subunit NOX2 and, thus, a functional ROS-forming NOX2 were obtained from The Jackson Laboratory. B6.129S7-Ifng^{tm1Ts}/J (Ifng^{-/-} or Ifng-KO) mice do not produce IFNγ (19). Naïve C57BL/6, Nox2-KO, and Ifng-KO mice (6-12 weeks of age) were treated intraperitoneally (i.p.) with PBS (control), HDC (Sigma, 1,500 μg/mouse), IL15 (0.04 μg/mouse), alone or combined, on the day before, the day after, and 3 days after intravenous (i.v.) inoculation of B16F10 cells (5×10⁴-15×10⁴ cells/mouse) or B16F1 cells (20×10⁴-30×10⁴ cells/mouse). Three weeks after tumor inoculation, mice were euthanized by cervical dislocation followed by harvesting of lungs and spleens. Lung metastasis was determined by counting visible pulmonary metastatic foci under a light microscope. The experimental design is outlined in FIG. 1A.

For assessment of the impact of NOX2 inhibition on immune parameters during the early phase of tumor progression, mice received HDC at 1,500 μg/mouse or PBS (control) 1 day before the inoculation of B16F10 cells followed by dissection of lungs at 30 minutes or 24 hours after tumor cell inoculation as shown in FIG. 2A. In the latter experiments, naïve mice and HDC-treated mice that did not receive melanoma cells were used as additional controls.

Preparation of Single-Cell Suspensions from Lungs and Spleens

Lung tissues were dissociated into single cells by combining enzymatic degradation of extracellular matrix with mechanical dissociation using gentle MACS Technology (Miltenyi Biotech) based on instructions provided by the manufacturer. Single-cell suspensions of splenocytes were prepared by mashing the spleens through a 70-μm cell strainer followed by depletion of erythrocytes using RBC Lysing buffer (Sigma-Aldrich).

### Flow Cytometry

The following fluorochrome-labeled antimouse mAbs were purchased from BD Biosciences: anti-CD45 (30-F11), anti-CD11c (HL3), anti-IaIe (2G9), anti-CD3 (145-2311), anti-CD4 (RM4-5), anti-CD8 (53-6.7), anti-NK1.1 (PK136), anti-CD19 (1D3), anti-CD11b (M1/70), anti-Gr1 (RB6-8C5), anti-CD40 (3/23), and anti-Ly6C (AL-21). Anti-CD33 (29A1.4) was from Ebiosciences; anti-F4/80 (BM8) and anti-CD69 (H1.2F3) were from BioLegend. LIVE/DEAD Fixable Yellow Dead Cell Stain Kit or DAPI (both from Invitrogen) were used as cell viability markers in flow cytometry analyses. A minimum of 100,000 gated live cells were collected on a four-laser BD LSRFortessa (405, 488, 532, and 640 nm). Data were analyzed using FACSDiva Version 8.0.1 software (BD Biosciences).

### Detection of ROS

Superoxide anion production was determined by use of the isoluminol-electrogenerated chemiluminescence technique as described elsewhere. Briefly, single-cell suspensions of lungs were diluted to 10⁷ cells/mL in Krebs-Ringer glucose buffer supplemented with isoluminol (10 mg/mL; Sigma-Aldrich) and horseradish peroxidase (HRP, 4 U/mL, Boehringer) and added to 96-well plates that were incubated at 37° C. Phorbol myristate acetate (PMA, 5×10⁻⁸ M, Sigma-Aldrich) or the formyl peptide receptor agonist Trp-Lys-Tyr-Met-Val-D-Met (WKYMVm) (10⁻⁵ M, Tocris Bioscience) were added for induction of ROS production. Light emission was recorded continuously using a FLUOstar Omega plate reader (BMG). In some experiments, HDC (10-1,000 μmol/L, final concentrations) was added 5 minutes prior to the addition of WKYMVm.

### Depletion of Gr1+ and NK Cells In Vivo

Gr1⁺ cells were depleted by i.p. injections of 400 μg anti-Gr1 antibody (BioXCell, Clone RB6-8C5) 2 days before B16 cell inoculation. This procedure depletes >95% of Gr1⁺ cells in blood and other tissues. NK cells were depleted by i.p. injections of 250 μg anti-NK1.1 antibody (BioXCell, Clone PK136) 4 days and 2 days before B16F10 cell inoculation. NK-cell depletion was confirmed by flow cytometry on lungs and spleen tissue harvested on days 1, 3, and 6 after antibody injection.

### NK-Cell Isolation and Adoptive Transfer

Spleens were harvested from WT C57BL/6 mice and single-cell suspensions were prepared. Splenocytes were enriched for NK cells by passage through nylon wool columns (Polysciences). NK cells were then negatively selected using an NK-cell isolation kit II (Miltenyi Biotech) according to the manufacturer's instructions to a purity of >70%. Five million enriched NK cells were injected i.v. 12 hours before inoculation of B16F10 cells. WT NK cells in Ifng^{-/-} mice were detected 2 days after adoptive transfer by collecting peripheral blood followed by DNA extraction and PCR. The primer pair used for detection of WT Ifng was 5′ AGAAGTAAGTGGAAGGGCCCAGAAG 3′ (SEQ ID NO:02) and 5′ AGGGAAACTGGGAGA GGAGAAATAT 3′ (SEQ ID NO:03). For detection of the disrupted IFNγ gene (Ifng−/−) the primer pair 5′ TCAGCGCAGGGGCGCCCGGTTCTTT 3′ (SEQ ID NO:04) and 5′ ATCGACAAGACCGGCTTCCATCCGA 3′ (SEQ ID NO:05) was used.

### Detection of IFNγ

Mice were pretreated with HDC (1,500 μg) or PBS on the day before i.v. inoculation of B16F10 cells. Thirty minutes after tumor cell inoculation mice were sacrificed and single-cell lung cell suspensions were prepared. Lung cells were cocultured overnight with B16 cells (500,000 cells/mL) in flat bottom 96-well plates at effector:target cell ratios of 1:1 to 50:1. Supernatants were collected after 24 hours and the IFNγ content was determined by ELISA (Mouse IFNγ DuoSet ELISA, R&D Systems).

### Statistical Analysis

Two-tailed paired or unpaired t tests were used for statistical calculations. For multiple comparisons, one-way ANOVA followed by the Holm-Sidak multiple-comparison test was used.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of preventing metastasis of a primary tumor in a subject, or treating or ameliorating a metastatic tumor in a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject.

2. The method of claim 1, wherein reducing the activity of NOX2 comprises administering an effective amount of a NOX2 inhibitor to the subject.

3. The method of claim 2, wherein the NOX2 inhibitor is selected from the group consisting of histamine dihydrochloride (HDC), histamine, N-methyl-histamine, 4-methyl-histamine, histamine phosphate, histamine diphosphate, GSK2795039, apocynin, GKT136901, GKT137831, ML171, VAS2870, VAS3947, celastrol, ebselen, perhexiline, grindelic acid, NOX2ds-tat, NOXAlds, fulvene-5, ACD 084, NSC23766, CAS 1177865-17-6, and CAS 1090893-12-1, and shionogi.

4. The method of claim 3, wherein the NOX2 inhibitor is HDC.

5. The method of claim 4, wherein reducing the expression level of a nucleic acid encoding NOX2 or the expression level of NOX2 protein in a cell comprises contacting the cell with an isolated nucleic acid selected from the group consisting of a guide RNA (gRNA), a small hairpin RNA (shRNA), a small interfering RNA (siRNA), a micro RNA (miRNA), an antisense polynucleotide, and a ribozyme.

6. The method of claim 5, wherein the isolated nucleic acid comprises a sequence encoding NOX2 or a fragment thereof, a sequence encoding antisense NOX2 or a fragment thereof, or an antisense nucleic acid complementary to a sequence encoding NOX2 or a fragment thereof.

7. The method of claim 6, wherein the isolated nucleic acid comprises a gRNA comprising a sequence complementary to the sequence of a target gene selected from the group consisting of NOX2, CYBA, NCF1, NCF2, NCF4, RAC1, and RAC2.

8. The method of claim 7, wherein the target gene is NOX2.

9. The method of claim 1, further comprising administering an additional therapeutic agent in combination with the NOX2 inhibitor or the isolated nucleic acid.

10. The method of claim 9, wherein the additional therapeutic agent is a NK cell activating agent.

11. The method of claim 10, wherein the NK cell activating agent is selected from the group consisting of IL-15, IFN-γ, IL-12, IL-18, IL-2, and CCL5.

12. The method of claim 1, wherein the additional therapeutic agent is IL-15 or IFN-γ.

13. (canceled)

14. (canceled)

15. (canceled)

16. The method of claim 1, wherein the primary or metastatic tumor is selected from the group consisting of a melanoma, a bladder cancer, a breast cancer, a pancreatic cancer, a colorectal cancer, a renal cancer, a prostate cancer, a stomach cancer, a thyroid cancer, a uterine cancer, and an ovarian cancer.

17. (canceled)

18. The method of claim 1, wherein the metastatic tumor comprises a melanoma, and wherein the melanoma is selected from the group consisting of lentigo maligna, lentigo maligna melanoma, superficial spreading melanoma, acral lentiginous melanoma, mucosal melanoma, nodular melanoma, polypoid melanoma, desmoplastic melanoma, melanoma with small nevus-like cells, melanoma with features of a Spitz nevus, uveal melanoma, and vaginal melanoma.

19. The method of claim 1, wherein the primary or metastatic tumor is located at a site selected from the group consisting of lung, liver, brain, peritoneum, adrenal gland, skin, muscle, vagina, and bone.

20. (canceled)

21. The method of claim 1, wherein the cell is a hematopoietic cell or a myeloid cell.

22. (canceled)

23. (canceled)

24. (canceled)

25. (canceled)

26. A method of increasing the level of natural killer (NK) cells in a metastatic tumor of a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject, wherein the level of NK cells in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

27. A method of decreasing the level of reactive oxygen species (ROS) in a metastatic tumor of a subject, the method comprising reducing the activity of nicotinamide adenine dinucleotide phosphate oxidase 2 (NOX2) or the expression level of a nucleic acid encoding NOX2 or the expression level of NOX2 protein in a cell of the subject, wherein the level of ROS in the metastatic tumor is increased compared to a metastatic tumor in an untreated subject in which the activity of NOX2 or the expression level of a nucleic acid encoding NOX2 or the expression level of NOX2 protein in a cell of the untreated subject has not been reduced.

28-76. (canceled)
